(19) United States
(12) Patent Application Publication
(10) Pub. No.: US 2022/0000832 A1
(43) Pub. Date: Jan. 6, 2022

(54) Pharmaceutical Composition for Preventing Greying of Hair and Preventing or Treating Poliosis or Vitiligo

(71) Applicant: INHA University Research and Business Foundation, Incheon (KR)

(72) Inventors: Eunki KIM, Seoul (KR); Jeonghyun SHIN, Incheon (KR); Dong-Gyu CHO, Incheon (KR); Hyangbok LEE, Incheon (KR); Sunju CHOI, Gimpo-si (KR); Gayeong KIM, Gunsan-si (KR)

(21) Appl. No.: 17/390,631

(22) Filed: Jul. 30, 2021

Related U.S. Application Data
(63) Continuation-in-part of application No. PCT/KR2020/001097, filed on Jan. 22, 2020.

(30) Foreign Application Priority Data
Feb. 1, 2019 (KR) 10-2019-0013868

(51) Int. Cl. A61K 31/341; A61P 17/00; A23L 33/10

(52) U.S. Cl. CPC A61K 31/341; A23L 33/10; A61P 17/00

(57) ABSTRACT

The present invention relates to a pharmaceutical composition for preventing greying of hair, and preventing or treating poliosis or vitiligo. The pharmaceutical composition of the present invention increases intracellular melanin content, accelerates or increases melanoblast migration, pre-emptively prevents gray hair formation, and accelerates dark hair formation, and thus may be beneficially used in the prevention or alleviation of greying of hair, and in the prevention, alleviation or treatment of poliosis or vitiligo.

### CROSS-REFERENCES TO RELATED APPLICATION

This application is a continuation-in-part of International Application No. PCT/KR2020/001097, filed on Jan. 22, 2020, which claims the benefit of Korean Application No. 10-2019-0013868, filed on Feb. 1, 2019, the contents of which are incorporated herein by reference.

### BACKGROUND OF THE INVENTION

### 1. Field of the Invention

The present invention relates to a pharmaceutical composition for preventing gray hair, and preventing or treating poliosis or vitiligo.

### 2. Description of the Related Art

Poliosis is the graying and whitening of hair due to a decrease in melanin produced by melanocytes in the hair, eyebrows, eyelashes, etc. The mechanism of poliosis is known to be due to a decrease in the number of melanocytes forming melanin pigment in hair follicles and the inability to move melanin to surrounding keratinocytes, resulting in pigment deficiency.

The upper part of the hair follicle extends from the epidermis to the deeper layers of the dermis as a tubular recessed form. The lower part of the hair follicle, or hair bulb, contains an invagination located in the papilla. The area around the papilla located at the bottom of the hair bulb is a region where matrix cells with a high degree of proliferation are distributed. These cells are the precursors to the keratinized cells that make up the hair. The cells made by proliferation of these precursors migrate vertically from the hair bulb, and the upper part of the hair bulb gradually becomes keratinized, and the keratinized cells gather to form a hair stalk.

The color of head hair and bodily hair is to some extent based on the amount and proportion of the two melanin groups: umelanins (brown and black pigments) and pheomelanins (red and yellow pigments). Pigmentation of head hair and bodily hair requires the presence of melanocytes in the hair bulb of the hair follicle. The melanin produced in melanocytes is transferred to keratinocytes to form hair stalks that make colored hair or hair strands. This structure is known as a “hair follicular pigmentation unit”.

Furthermore, at least three enzymes, tyrosinase, DOPA chrome tautomerase (TRP-2) and DHICA oxidase (TRP-1), are involved in the production of melanin in mammals. The activity of these three enzymes is known to be essential for maximal activation of melanin biosynthesis.

First, the tyrosinase is referred to as an enzyme that initiates the biosynthesis of melanin or limits the formation of melanin. In addition, tyrosinase catalyzes the oxidation of tyrosine to DOPA and then to dopaquinone. Dopaquinone compounds are naturally transformed into dopachrome or cysteinyldopa derivatives in the presence of cysteine.

The TRP-2 catalyzes the tautomerization of DOPA chrome to 5,6-dihydroxyindole-2-carboxylic acid (DHICA). In the presence of TRP-2, DOPA chrome undergoes spontaneous decarboxylation to form 5,6-dihydroxyindole (DHI). In addition, the TRP-1 oxidizes the DHICA compound to form a quinone derivative.

Poliosis is affected by both melanocytes in the hair root and precursors of melanocytes, and is associated with a specific and progressive depletion of hair melanocytes. Other types of cells present in the hair follicle are not affected. In addition, this depletion of melanocytes is not observed in the epidermis. The cause of this gradual and specific depletion of melanocytes and melanocyte precursors in the hair follicle has not yet been identified.

In addition, head hair and bodily hair have a cycle. This cycle consists of an anagenic phase, a catagenic phase, and a telogenic phase, and then proceeds to a new growth period. As a result of this hair cycle, the hair follicular pigmentation unit must also periodically resume. During the transition from catagenic phase to anagenic phase in humans, some inactive melanocytes proliferate and begin to express enzymes necessary for melanin synthesis such as tyrosinase and TRP-1, except for TRP-2, located around the papilla of the hair bulb in the developmental stage. Along with this, in the upper layer of the hair follicle, the remaining quiescent melanocytes remain inactive. Tyrosinase and TRP-1 are expressed in melanocytes of the hair bulb during the anagenic phase, but are no longer expressed in melanocytes during the catagenic phase and telogen. Therefore, the normal cycle of melanocytes in the human hair follicle requires the presence of melanocyte precursors that can be activated periodically to regenerate the hair follicular pigmentation unit, which is present in the upper part of the hair follicle.

European Patent Publication EP 1870081 discloses a composition for treating poliosis comprising ellagic acid or a derivative thereof as an active ingredient.

On the other hand, vitiligo is an acquired depigmentation disease in which depigmented spots with various sizes and shapes appear on the skin due to the loss of melanocytes. Vitiligo occurs in 0.1%-0.2% of the world's population in various ways, and it can be a cosmetic problem for patients and can cause serious mental problems such as difficulties in interpersonal relationships. Histologically, loss of epidermal melanocytes appears in the discolored area of vitiligo, and the cause is still unknown, but autoimmune, neurological, self-destruction, stress, and viral hypotheses are being discussed.

Several methods have been used to treat vitiligo. Vitiligo patients with discolored areas are often successfully treated with 8-methoxypsoralen (8-MOP) and ultraviolet (UV) A radiation (PUVA therapy). In this treatment, repigmentation occurs well in the areas where the hair follicles are concentrated, and the repigmentation occurs slowly. When re-pigmentation occurs in the areas where the hair follicles are concentrated, melanoblasts, the precursors of melanocytes, are present at the ends of the hair follicles. In patients with vitiligo, the activity of epidermal melanocytes is lost, but the melanocytes of the outer hair root sheath of the hair follicle are not affected. As such, the presence of the inactive melanocytes is suggested as a possibility to induce repigmentation in vitiligo patients. An effective therapeutic agent has been proposed as a substance that promotes differentiation, proliferation, and migration of inactive melanoblasts along the surface of the outer hair root sheath near the epidermis. Melanoblasts are non-depigmented cells and are defined as precursors of melanocytes. Melanoblasts lack tyrosinase and do not stain with DOPA, or produce melanin. Therefore, melanocytes are provided as an ideal model for understanding the effects of natural substances and the characteristics of their mechanisms for differentiation into melanocytes. Korean Patent Publication No. 1020120003649 discloses a composition for preventing gray hair and treating vitiligo comprising Broussometia kazinoki extract.

### SUMMARY OF THE INVENTION

It is an object of the present invention to provide a pharmaceutical composition for preventing gray hair, and preventing or treating poliosis or vitiligo.

To achieve the above object, in an aspect of the present invention, the present invention provides a pharmaceutical composition for preventing or treating poliosis or vitiligo comprising a compound represented by formula 1 below, an isomer thereof, a solvate thereof, a hydrate thereof, or a pharmaceutically acceptable salt thereof as an active ingredient:

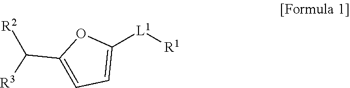

In formula 1,

L¹, R¹, R² and R³ are as defined in this specification.

In another aspect of the present invention, the present invention provides a cosmetic composition for preventing or alleviating poliosis or vitiligo comprising a compound represented by formula 1 below, an isomer thereof, a solvate thereof, a hydrate thereof, or a pharmaceutically acceptable salt thereof as an active ingredient:

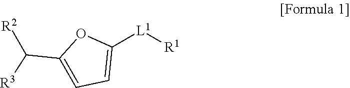

In formula 1,

L¹, R¹, R² and R³ are as defined in this specification.

In another aspect of the present invention, the present invention provides a pharmaceutical composition for preventing or alleviating gray hair comprising a compound represented by formula 1 below, an isomer thereof, a solvate thereof, a hydrate thereof, or a pharmaceutically acceptable salt thereof as an active ingredient:

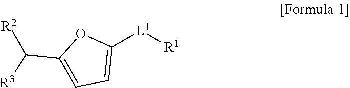

In formula 1,

L¹, R¹, R² and R³ are as defined in this specification.

In another aspect of the present invention, the present invention provides a cosmetic composition for preventing or alleviating gray hair comprising a compound represented by formula 1 below, an isomer thereof, a solvate thereof, a hydrate thereof, or a pharmaceutically acceptable salt thereof as an active ingredient:

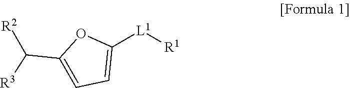

In formula 1,

L¹, R¹, R² and R³ are as defined in this specification.

In another aspect of the present invention, the present invention provides a health functional food composition for preventing or alleviating poliosis or vitiligo comprising a compound represented by formula 1 below, an isomer thereof, a solvate thereof, a hydrate thereof, or a pharmaceutically acceptable salt thereof as an active ingredient:

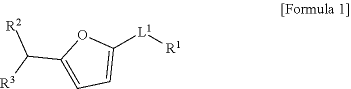

In formula 1,

L¹, R¹, R² and R³ are as defined in this specification.

In another aspect of the present invention, the present invention provides a health functional food composition for preventing or alleviating gray hair comprising a compound represented by formula 1 below, an isomer thereof, a solvate thereof, a hydrate thereof, or a pharmaceutically acceptable salt thereof as an active ingredient:

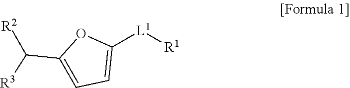

In formula 1,

L¹, R¹, R² and R³ are as defined in this specification.

In another aspect of the present invention, the present invention provides a method for preventing or treating poliosis or vitiligo comprising a step of administering the pharmaceutical composition or health functional food composition comprising a compound represented by formula 1 below, an isomer thereof, a solvate thereof, a hydrate thereof, or a pharmaceutically acceptable salt thereof as an active ingredient to a subject in need:

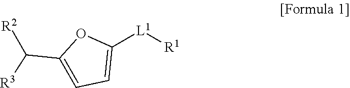

In formula 1,

L¹, R¹, R² and R³ are as defined in this specification.

In another aspect of the present invention, the present invention provides a use of the pharmaceutical composition or health functional food composition comprising a compound represented by formula 1 below, an isomer thereof, a solvate thereof, a hydrate thereof, or a pharmaceutically acceptable salt thereof as an active ingredient in the prevention or treatment of poliosis or vitiligo:

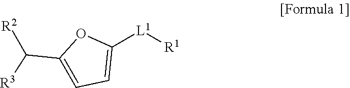

In formula 1,

L¹, R¹, R² and R³ are as defined in this specification.

### Advantageous Effect

The pharmaceutical composition of the present invention increases intracellular melanin content, accelerates or increases melanoblast migration, pre-emptively prevents gray hair formation, and accelerates dark hair formation, and thus may be beneficially used in the prevention or alleviation of gray hair, and in the prevention, alleviation or treatment of poliosis or vitiligo.

### BRIEF DESCRIPTION OF THE DRAWINGS

FIG. 1 is a graph showing the results of evaluating the cell viability according to the treatment concentrations of the compound of Example 1.

FIG. 2 is a graph showing the results of evaluating the cell viability according to the treatment of the compounds of Examples 2-10 at the concentration of 10 μM.

FIG. 3 is a graph showing the results of evaluating the cell viability according to the treatment concentrations of the compounds of Examples 9 and 10.

FIG. 4 is a graph showing the results of evaluating the cell viability according to the treatment of the compounds of Examples 11-18 at the concentration of 10 μM.

FIG. 5 is a graph showing the results of evaluating the cell viability according to the treatment concentrations of the compound of Example 18.

FIG. 6 is a graph showing the results of evaluating the cell viability according to the treatment of the compounds of Examples 19-21 at the concentration of 10 μM.

FIG. 7 is a graph showing the results of evaluating the intracellular melanin content according to the treatment concentrations of the compound of Example 1.

FIG. 8 is a graph showing the results of evaluating the intracellular melanin content according to the treatment of the compounds of Examples 2-10 at the concentration of 10 μM.

FIG. 9 is a graph showing the results of evaluating the intracellular melanin content according to the treatment concentrations of the compounds of Examples 9 and 10.

FIG. 10 is a graph showing the results of evaluating the intracellular melanin content according to the treatment of the compounds of Examples 11-18 at the concentration of 10 μM.

FIG. 11 is a graph showing the results of evaluating the intracellular melanin content according to the treatment concentrations of the compound of Example 18.

FIG. 12 is a graph showing the results of evaluating the intracellular melanin content according to the treatment of the compounds of Examples 19-21 at the concentration of 10 μM.

FIG. 13 is a graph showing the results of evaluating the melanoblast migration rate according to the treatment concentrations (1-1000 μM) of the compound of Example 1.

FIG. 14 is a set of photographs taken at 40 times magnification under a microscope after dividing the filter into quarters to measure the number of cells in the transwell migration assay performed to confirm the melanoblast migration according to the treatment concentrations (1-1000 μM) of the compound of Example 1.

FIG. 15 is a graph showing the results of evaluating the melanoblast migration rate according to the treatment concentrations (10-25 μM) of the compound of Example 1.

FIG. 16 is a set of photographs taken at 40 times magnification under a microscope after dividing the filter into quarters to measure the number of cells in the transwell migration assay performed to confirm the melanoblast migration according to the treatment concentrations (10-25 μM) of the compound of Example 1.

FIG. 17 is a graph showing the results of evaluating the melanoblast migration rate according to the treatment of the compounds of Examples 2-10 at the concentration of 10 μM.

FIG. 18 is a set of photographs taken at 40 times magnification under a microscope after dividing the filter into quarters to measure the number of cells in the transwell migration assay performed to confirm the melanoblast migration according to the treatment of the compounds of Examples 2-10 at the concentration of 10 μM.

FIG. 19 is a graph showing the results of evaluating the melanoblast migration rate according to the treatment concentrations of the compounds of Examples 9 and 10.

FIG. 20 is a set of photographs taken at 40 times magnification under a microscope after dividing the filter into quarters to measure the number of cells in the transwell migration assay performed to confirm the melanoblast migration according to the treatment concentrations of the compounds of Examples 9 and 10.

FIG. 21 is a graph showing the results of evaluating the melanoblast migration rate according to the treatment of the compounds of Examples 11-18 at the concentration of 10 μM.

FIG. 22 is a set of photographs taken at 40 times magnification under a microscope after dividing the filter into quarters to measure the number of cells in the transwell migration assay performed to confirm the melanoblast migration according to the treatment of the compounds of Examples 11-18 at the concentration of 10 μM.

FIG. 23 is a graph showing the results of evaluating the melanoblast migration rate according to the treatment concentrations of the compound of Example 18.

FIG. 24 is a set of photographs taken at 40 times magnification under a microscope after dividing the filter into quarters to measure the number of cells in the transwell migration assay performed to confirm the melanoblast migration according to the treatment concentrations of the compound of Example 18.

FIG. 25 is a graph showing the results of evaluating the melanoblast migration rate according to the treatment of the compounds of Examples 19-21 at the concentration of 10 μM.

FIG. 26 is a set of photographs taken at 40 times magnification under a microscope after dividing the filter into quarters to measure the number of cells in the transwell migration assay performed to confirm the melanoblast migration according to the treatment of the compounds of Examples 19-21 at the concentration of 10 μM.

### DETAILED DESCRIPTION OF THE INVENTION

Hereinafter, the present invention is described in detail.

In an aspect of the present invention, the present invention provides a pharmaceutical composition for preventing or treating poliosis or vitiligo comprising a compound represented by formula 1 below, an isomer thereof, a solvate thereof, a hydrate thereof, or a pharmaceutically acceptable salt thereof as an active ingredient:

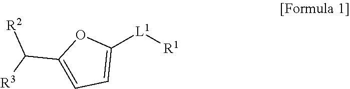

In formula 1,

L¹ is —O(═O)—, straight or branched [symbol] C₁₋₅ alkylene, —C(═O)O— or —O(═O)NH—, R¹ is hydrogen, OH, straight or branched C₁₋₅ alkyl, straight or branched C₁₋₅ alkylcarbonyloxy, C₆₋₁₀ aryl-C₁₋₂ alkyl, allyloxy or C₆₋₁₀ aryl-C₁₋₂ alkyloxy; and

R² and R³ are independently hydrogen, OH, straight or branched C₁₋₅ alkyl or straight or branched C₁₋₅ alkoxy, or, R² and R³ can form carbonyl (C═O) along with the carbon atom to which they are attached, except when R² and R³ are hydrogen at the same time.

In formula 1,

L¹ is —O(═O)—, straight or branched [symbol] C₁₋₃ alkylene, —C(═O)O— or —C(═O)NH—, R¹ is hydrogen, OH, straight or branched C₁₋₃ alkyl, straight or branched C₁₋₃ alkylcarbonyloxy, phenyl-C₁₋₂ alkyl, allyloxy or phenyl-C₁₋₂ alkyloxy; and

R² and R³ are independently hydrogen, OH, straight or branched C₁₋₃ alkyl or straight or branched C₁₋₃ alkoxy, or, R² and R³ can form carbonyl (C═O) along with the carbon atom to which they are attached, except when R² and R³ are hydrogen at the same time.

In formula 1,

L¹ is —C(═O)—, —CH₂—, —C(═O)O— or —C(═O)NH—, R¹ is hydrogen, OH, methyl, ethyl, methylcarbonyloxy, benzyl, allyloxy or benzyloxy; and

R² and R³ are independently hydrogen, OH, methyl or methoxy, or, R² and R³ can form carbonyl (C═O) along with the carbon atom to which they are attached, except when R² and R³ are hydrogen at the same time.

Preferable examples of the compound represented by formula 1 according to the present invention include the following compounds:
<1> 5-(hydroxymethyl)furan-2-carbaldehyde;
<2> 5-((benzyloxy)methyl)furan-2-carbaldehyde;
<3> 5-((allyloxy)methyl)furan-2-carbaldehyde;
<4> N-benzyl-5-formylfuran-2-carboxamide;
<5> 5-formyl-N-propylfuran-2-carboxamide;
<6> 5-formyl-N-methylfuran-2-carboxamide;
<7> benzyl 5-formylfuran-2-carboxylate;
<8> allyl 5-formylfuran-2-carboxylate;
<9> methyl 5-(dimethoxymethyl)furan-2-carboxylate;
<10> methyl 5-formylfuran-2-carboxylate;
<11> ethyl 5-(hydroxymethyl)furan-2-carboxylate;
<12> furan-2,5-diyldimethanol;
<13> methyl 5-(hydroxymethyl)furan-2-carboxylate;
<14> 5-(hydroxymethyl)furan-2-carboxylic acid;
<15> 1-(5-(((tetrahydro-2H-pyran-2-yl)oxy)methyl)furan-2-yl)ethanol;
<16> 1-(5-(hydroxymethyl)furan-2-yl)ethanol;
<17> furan-2,5-dicarbaldehyde;
<18> 5-(1-hydroxyethyl)furan-2-carbaldehyde;
<19> 1-(5-formylfuran-2-yl)ethyl acetate;
<20> 5-(1-hydroxypropyl)furan-2-carbaldehyde; and
<21> 1-(5-formylfuran-2-yl)propyl acetate.

The composition increases intracellular melanin content, accelerates or increases melanoblast migration, pre-emptively prevents gray hair formation, and accelerates dark hair formation, and thus may be beneficially used in the prevention, alleviation or treatment of poliosis or vitiligo.

The compound represented by formula 1 of the present invention is contained in plants such as apple mango, chicory, plum, Melia azedarach, apricot, and castor-oil plant.

The compound represented by formula 1 of the present invention can be used as a form of a pharmaceutically acceptable salt, in which the salt is preferably acid addition salt formed by pharmaceutically acceptable free acids. The acid addition salt herein can be obtained from inorganic acids such as hydrochloric acid, nitric acid, phosphoric acid, sulfuric acid, hydrobromic acid, hydroiodic acid, nitrous acid, and phosphorous acid; non-toxic organic acids such as aliphatic mono and dicarboxylate, phenyl-substituted alkanoate, hydroxy alkanoate and alkandioate, aromatic acids, and aliphatic and aromatic sulfonic acids; and organic acids such as trifluoroacetic acid, acetate, benzoic acid, citric acid, lactic acid, maleic acid, gluconic acid, methanesulfonic acid, 4-toluenesulfonic acid, tartaric acid, and fumaric acid, etc. The pharmaceutically non-toxic salts are exemplified by sulfate, pyrosulfate, bisulfate, sulphite, bisulphite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutylate, caprate, heptanoate, propiolate, oxalate, malonate, succinate, suberate, cabacate, fumarate, maliate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutylate, citrate, lactate, β-hydroxybutylate, glycolate, malate, tartrate, methanesulfonate, propanesulfonate, naphthalene-1-sulfonate, naphthalene-2-sulfonate, and mandelate.

The acid addition salt according to the present invention can be prepared by the conventional method known to those in the art. For example, the derivative represented by formula 1 is dissolved in an organic solvent such as methanol, ethanol, acetone, methylene chloride, and acetonitrile, to which organic acid or inorganic acid is added to induce precipitation. Then, the precipitate is filtered and dried to give the salt. Or the solvent and the excessive acid are distillated under reduced pressure, and dried to give the salt. Or the precipitate is crystallized in an organic solvent to give the same.

A pharmaceutically acceptable metal salt can be prepared by using a base. Alkali metal or alkali earth metal salt is obtained by the following processes: dissolving the compound in excessive alkali metal hydroxide or alkali earth metal hydroxide solution; filtering non-soluble compound salt; evaporating the remaining solution and drying thereof. At this time, the metal salt is preferably prepared in the pharmaceutically suitable form of sodium, potassium, or calcium salt. And the corresponding silver salt is prepared by the reaction of alkali metal or alkali earth metal salt with proper silver salt (ex; silver nitrate).

In addition, the present invention includes not only the compound represented by formula 1 but also a pharmaceutically acceptable salt thereof, and a solvate, an optical isomer, or a hydrate possibly produced from the same.

The term “hydrate” refers to the compound of the present invention or the salt thereof comprising a stoichiometric or non-stoichiometric amount of water bound by non-covalent intermolecular force. The hydrate of the compound represented by formula 1 of the present invention can include a stoichiometric or non-stoichiometric amount of water that is bound by non-covalent intermolecular force. The hydrate can contain more than 1 equivalent of water, preferably, 1 to 5 equivalents of water. Such a hydrate can be prepared by crystallizing the compound represented by formula 1 of the present invention, the isomer thereof, or the pharmaceutically acceptable salt thereof from water or a solvent containing water.

The term “solvate” refers to the compound of the present invention or the salt thereof comprising a stoichiometric or non-stoichiometric amount of a solvent bound by non-covalent intermolecular force. Preferred solvents therefor include solvents that are volatile, non-toxic, and/or suitable for administration to humans.

The term “isomer” refers to the compound of the present invention or the salt thereof having the same chemical or molecular formula but different structurally or sterically. Such isomers include structural isomers such as tautomers, stereoisomers such as geometric isomers (trans, cis), and optical isomers (enantiomers). All these isomers and mixtures thereof are also included in the scope of the present invention.

The compound represented by formula 1 or the pharmaceutically acceptable salt thereof can be administered orally or parenterally in various formulations at the time of clinical administration. In the case of formulation, it is prepared using generally used diluents or excipients such as fillers, extenders, binders, wetting agents, disintegrating agents and surfactants. Solid formulations for oral administration are tablets, pills, powders, granules and capsules. These solid formulations are prepared by mixing the compound represented by formula 1 or the pharmaceutically acceptable salt thereof of the present invention with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, and emulsions. Water insoluble excipients and suspensions can contain propylene glycol, polyethylene glycol, vegetable oil like olive oil, injectable ester like ethylolate, etc.

The pharmaceutical composition comprising the compound represented by formula 1 or the pharmaceutically acceptable salt thereof as an active ingredient can be administered by parenterally and the parenteral administration includes subcutaneous injection, intravenous injection, intramuscular injection, or intrathoracic injection.

To prepare the compound represented by formula 1 or the pharmaceutically acceptable salt thereof as a formulation for parenteral administration, the compound represented by formula 1 or the pharmaceutically acceptable salt thereof is mixed with a stabilizer or a buffering agent in water to produce solution or suspension, which is then formulated as ampoules or vials. The composition herein can be sterilized and additionally contains preservatives, stabilizers, wettable powders or emulsifiers, salts and/or buffers for the regulation of osmotic pressure, and other therapeutically useful materials, and the composition can be formulated by the conventional mixing, granulating or coating method.

The formulations for oral administration are exemplified by tablets, pills, hard/soft capsules, solutions, suspensions, emulsions, syrups, granules, elixirs, and troches, etc. These formulations can include diluents (for example, lactose, dextrose, sucrose, mannitol, sorbitol, cellulose, and/or glycine) and lubricants (for example, silica, talc, stearate and its magnesium or calcium salt, and/or polyethylene glycol) in addition to the active ingredient. Tablets can include binding agents such as magnesium aluminum silicate, starch paste, gelatin, methylcellulose, sodium carboxymethylcellulose and/or polyvinylpyrolidone, and if necessary disintegrating agents such as starch, agarose, alginic acid or its sodium salt or azeotropic mixtures and/or absorbents, coloring agents, flavours, and sweeteners can be additionally included thereto.

In another aspect of the present invention, the present invention provides a cosmetic composition for preventing or alleviating poliosis or vitiligo comprising a compound represented by formula 1 below, an isomer thereof, a solvate thereof, a hydrate thereof, or a pharmaceutically acceptable salt thereof as an active ingredient:

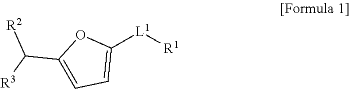

L¹, R¹, R² and R³ are as defined in formula 1 of the pharmaceutical composition for preventing or treating poliosis or vitiligo.

The composition increases intracellular melanin content, accelerates or increases melanoblast migration, pre-emptively prevents gray hair formation, and accelerates dark hair formation, and thus may be beneficially used as a cosmetic composition for preventing or alleviating poliosis or vitiligo.

In preparing the cosmetic composition of the present invention, 3 to 30 weight parts, preferably 5 to 20 weight parts, of the compound represented by formula 1 of the present invention can be added to the cosmetic composition usually contained therein.

In addition, the cosmetic composition comprising the compound represented by formula 1 of the present invention can additionally include a supplement generally used in the field of skin science such as fatty substance, organic solvent, resolvent, concentrate, gelling agent, softener, antioxidant, suspending agent, stabilizer, foaming agent, odorant, surfactant, water, ionic or non-ionic emulsifying agent, filler, sequestering agent, chelating agent, preserving agent, vitamin, blocker, moisturing agent, essential oil, dye, pigment, hydrophilic or hydrophobic activator, lipid vesicle or other components generally used in a preparation for skin external application. The amount of the above supplement can be determined as generally accepted in the field of skin science.

The cosmetic composition according to the present invention can be prepared in a formulation selected from the group consisting of solutions, external ointments, creams, foams, nourishing lotions, softening lotions, packs, soft water, emulsions, makeup bases, essences, soaps, liquid detergents, bath agents, sunscreen creams, sun oils, suspensions, emulsions, pastes, gels, lotions, powders, soaps, surfactant-containing cleansing, oils, powdered foundations, emulsified foundations, wax foundations, patches and sprays, but not always limited thereto.

In addition, the cosmetic composition of the present invention can further include one or more cosmetically acceptable carriers to be formulated in general skin cosmetics, and as common ingredients, for example, oil, water, surfactant, humectant, lower alcohol, thickener, chelating agent, colorant, preservative, fragrance, etc. can be appropriately mixed, but not always limited thereto.

The cosmetically acceptable carrier included in the cosmetic composition of the present invention varies depending on the formulation. In the case that the cosmetic composition is formulated as ointment, paste, cream or gel, the proper carrier can be selected from the group consisting of animal oil, vegetable oil, wax, paraffin, starch, tracanth, cellulose derivative, polyethylene glycol, silicon, bentonite, silica, talk, zinc oxide and mixtures thereof.

In the case that the cosmetic composition is formulated as powder or spray, the proper carrier can be selected from the group consisting of lactose, talc, silica, aluminum hydroxide, calcium silicate, polyamide powder and mixtures thereof, and in particular if the composition of the present invention is formulated as spray, a propellant such as chlorofluorohydrocarbon, propane/butane or dimethyl ether can be additionally included.

In the case that the cosmetic composition is formulated as liquid or emulsion, the proper carrier can be selected from the group consisting of solvent, solubilizer and emulsifier, which is exemplified by water, ethanol, isopropanol, ethyl carbonate, ethyl acetate, benzyl benzoate, propylene glycol, and 1,3-butylglycol oil. In particular, examples of the carrier include cottonseed oil, peanut oil, corn germ oil, olive oil, castor oil and sesame oil, glycerol aliphatic ester, polyethylene glycol and fatty acid ester of sorbitan.

In the case that the cosmetic composition is formulated as suspension, the proper carrier can be selected from the group consisting of liquid diluent such as water, ethanol or propylene glycol; suspending agent such as ethoxylated isostearyl alcohol, polyoxyethylene sorbitol ester and polyoxyethylene sorbitan ester; microcrystalline cellulose; aluminum methahydroxide; bentonite; agar; and tragacanth.

In the case that the cosmetic composition is formulated as soap, the proper carrier can be selected from the group consisting of alkali metal salt of fatty acid, fatty acid hemiester salt, fatty acid protein hydrolysate, isethionate, lanolin derivative, aliphatic alcohol, vegetable oil, glycerol, sugars, etc.

In another aspect of the present invention, the present invention provides a health functional food composition for preventing or alleviating poliosis or vitiligo comprising a compound represented by formula 1 below, an isomer thereof, a solvate thereof, a hydrate thereof, or a pharmaceutically acceptable salt thereof as an active ingredient:

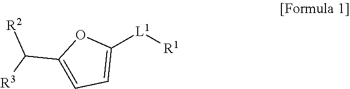

L¹, R¹, R² and R³ are as defined in formula 1 of the pharmaceutical composition for preventing or treating poliosis or vitiligo.

The composition increases intracellular melanin content, accelerates or increases melanoblast migration, pre-emptively prevents gray hair formation, and accelerates dark hair formation, and thus may be beneficially used as a health functional food composition for preventing or alleviating poliosis or vitiligo.

The compound represented by formula 1 according to the present invention can be added as it is or as mixed with other food components according to the conventional method. The mixing ratio of active ingredients can be regulated according to the purpose of use (prevention or alleviation). In general, the compound included in the health food can be added in an amount of 0.1 to 90 weight parts based on the total food weight. However, if long term administration is required for health and hygiene or regulating health condition, the content can be lower than the above but higher content can be accepted as well since the compound has been proved to be very safe.

The composition for health beverages of the present invention can additionally include various flavors or natural carbohydrates, etc, like other beverages in addition to the compound. The natural carbohydrates above can be one of monosaccharides such as glucose and fructose; disaccharides such as maltose and sucrose; polysaccharides such as dextrin and cyclodextrin; and sugar alcohols such as xilytole, sorbitol and erythritol. Besides, natural sweetening agents (thaumatin, stevia extract, for example rebaudioside A, glycyrrhizin, etc.) and synthetic sweetening agents (saccharin, aspartame, etc.) can be included as a sweetening agent. The content of the natural carbohydrate is preferably 1-20 g and more preferably 5-12 g in 100 g of the composition of the present invention.

In addition to the ingredients mentioned above, the compound represented by formula 1 according to the present invention can include in variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. The compound represented by formula 1 of the present invention can also include fruit flesh for the preparation of natural fruit juice, fruit beverages and vegetable beverages.

In another aspect of the present invention, the present invention provides a pharmaceutical composition for preventing or alleviating gray hair comprising a compound represented by formula 1 below, an isomer thereof, a solvate thereof, a hydrate thereof, or a pharmaceutically acceptable salt thereof as an active ingredient:

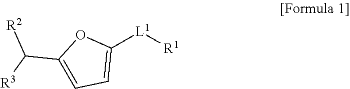

L¹, R¹, R² and R³ are as defined in formula 1 of the pharmaceutical composition for preventing or treating poliosis or vitiligo.

The composition increases intracellular melanin content, accelerates or increases melanoblast migration, pre-emptively prevents gray hair formation, and accelerates dark hair formation, and thus may be beneficially used as a pharmaceutical composition for preventing or alleviating gray hair.

The detailed description of the pharmaceutical composition for preventing or alleviating gray hair is the same as the description of the pharmaceutical composition for preventing or treating poliosis or vitiligo.

In another aspect of the present invention, the present invention provides a cosmetic composition for preventing or alleviating gray hair comprising a compound represented by formula 1 below, an isomer thereof, a solvate thereof, a hydrate thereof, or a pharmaceutically acceptable salt thereof as an active ingredient:

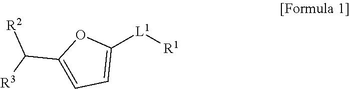

L¹, R¹, R² and R³ are as defined in formula 1 of the pharmaceutical composition for preventing or treating poliosis or vitiligo.

The composition increases intracellular melanin content, accelerates or increases melanoblast migration, pre-emptively prevents gray hair formation, and accelerates dark hair formation, and thus may be beneficially used as a cosmetic composition for preventing or alleviating gray hair.

The detailed description of the cosmetic composition for preventing or alleviating gray hair is the same as the description of the cosmetic composition for preventing or alleviating poliosis or vitiligo.

In another aspect of the present invention, the present invention provides a health functional food composition for preventing or alleviating gray hair comprising a compound represented by formula 1 below, an isomer thereof, a solvate thereof, a hydrate thereof, or a pharmaceutically acceptable salt thereof as an active ingredient:

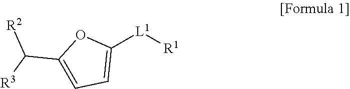

L¹, R¹, R² and R³ are as defined in formula 1 of the pharmaceutical composition for preventing or treating poliosis or vitiligo.

The composition increases intracellular melanin content, accelerates or increases melanoblast migration, pre-emptively prevents gray hair formation, and accelerates dark hair formation, and thus may be beneficially used as a health functional food composition for preventing or alleviating gray hair.

The detailed description of the health functional food composition for preventing or alleviating gray hair is the same as the description of the health functional food composition for preventing or alleviating poliosis or vitiligo.

In another aspect of the present invention, the present invention provides a method for preventing or treating poliosis or vitiligo comprising a step of administering the pharmaceutical composition or health functional food composition comprising a compound represented by formula 1 below, an isomer thereof, a solvate thereof, a hydrate thereof, or a pharmaceutically acceptable salt thereof as an active ingredient to a subject in need:

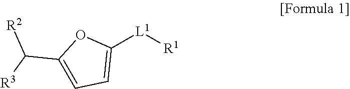

L¹, R¹, R² and R³ are as defined in formula 1 of the pharmaceutical composition for preventing or treating poliosis or vitiligo.

In another aspect of the present invention, the present invention provides a use of the pharmaceutical composition or health functional food composition comprising a compound represented by formula 1 below, an isomer thereof, a solvate thereof, a hydrate thereof, or a pharmaceutically acceptable salt thereof as an active ingredient in the prevention or treatment of poliosis or vitiligo:

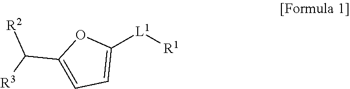

L¹, R¹, R² and R³ are as defined in formula 1 of the pharmaceutical composition for preventing or treating poliosis or vitiligo.

Hereinafter, the present invention will be described in detail by the following examples and experimental examples.

However, the following examples and experimental examples are only for illustrating the present invention, and the contents of the present invention are not limited thereto.

### Example 1: Preparation of 5-(hydroxymethyl)furan-2-carbaldehyde

5-(Hydroxymethyl)furan-2-carbaldehyde of Example 1 was purchased from Tokyo Chemical Industry Co., LTD. and used.

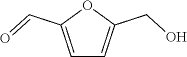

CAS RN: 67-47-0,

Product No: 67-47-0

The compounds of Examples 2 to 21 were prepared according to the methods shown in reaction formula A or B below, and specific preparation methods are described in each Example.

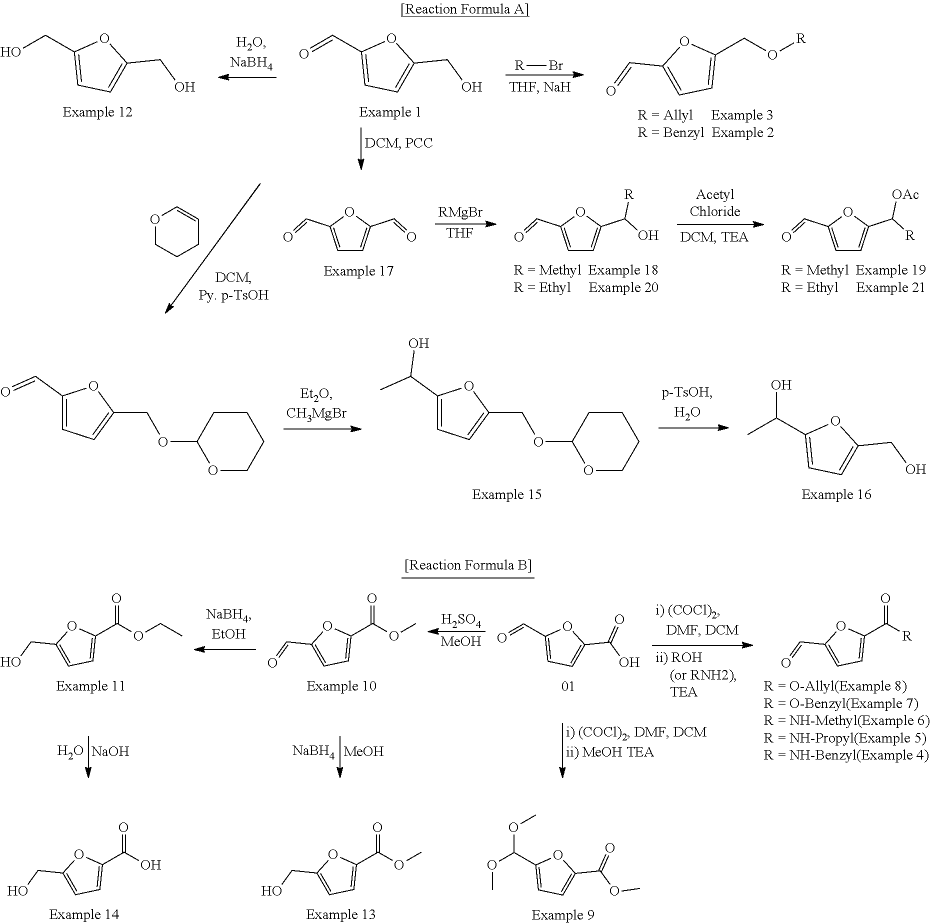

### Example 2: Preparation of 5-((benzyloxy)methyl)furan-2-carbaldehyde

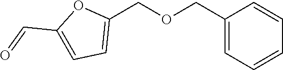 [SMILES: O=Cc1ccc(COCc2ccccc2)o1]

5-Hydroxylfurfural (100 mg, 0.79 mmol) and benzyl bromide (0.113 ml, 0.95 mmol) were dissolved in DMF (1.5 ml) under inert conditions. This solution was cooled to 0° C., to which NaH (38 mg, 0.95 mmol) was added. The mixture was stirred at room temperature for 5 hours and dried by evaporation. The reactant was diluted in diethyl ether, and the organic layer was washed with water. The organic layer was dried over Na₂SO₄ and dried by evaporation. The residue was purified by silica gel chromatography (ethyl acetate:hexane=1:4) to give a target compound (66.7 mg, 39%).

¹H NMR (400 MHz, CDCl₃) δ 9.53 (s, 1H), 7.24 (m, 5H) 7.12 (d, J=3.5 Hz, 1H), 6.44 (d, J=3.5 Hz, 1H), 4.51 (s, 1H), 4.48 (s, 1H); ¹³C NMR (101 MHz, CDCl₃) δ 177.81, 158.49, 152.73, 137.33, 128.62, 128.10, 128.00, 127.05, 111.38, 73.00, 64.20.

### Example 3: Preparation of 5-((allyloxy)methyl)furan-2-carbaldehyde

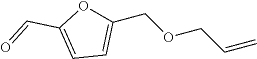

A target compound was obtained (13.22 mg, 10%) by performing a method similar to that of Example 2.

¹H NMR (400 MHz, CDCl₃) δ 9.63 (s, 1H), 7.21 (d, J=3.5 Hz, 1H), 6.53 (d, J=3.5 Hz, 1H), 5.92 (ddt, J=17.2, 10.4, 5.7 Hz, 1H), 5.32 (dq, J=17.2, 1.6 Hz, 1H), 5.24 (dq, J=10.4, 1.6 Hz, 1H), 4.55 (s, 2H), 4.08 (dt, J=5.7, 1.6 Hz, 2H); ¹³C NMR (101 MHz, CDCl₃) δ 177.80, 158.53, 152.69, 133.91, 121.94, 118.13, 111.25, 71.91, 64.12.

### Example 4: Preparation of N-benzyl-5-formylfuran-2-carboxamide

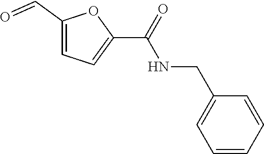 [SMILES: O=Cc1ccc(C(=O)NCc2ccccc2)o1]

5-Formylfuran-2-carboxylic acid (100 mg, 0.71 mmol) was dissolved in DMF (2 ml) under inert conditions. After oxalyl chloride (0.092 ml, 1.07 mmol) was added dropwise thereto, DMF was added dropwise until foaming occurred. The mixture was stirred at room temperature for 1 hour. This solution was cooled to 0° C., to which benzyl amine (0.23 ml, 2.14 mmol) and TEA (0.3 ml, 2.14 mmol) were added. The solution was stirred at 0° C. for 2 hours and dried by evaporation. The reactant was diluted in DCM, and the organic layer was washed with water and 1 M HCl solution. The organic layer was dried over Na₂SO₄ and dried by evaporation. The residue was purified by silica gel chromatography (ethyl acetate:hexane=1:1) to give a target compound (115 mg, 70%).

¹H NMR (400 MHz, CDCl₃) δ 9.64 (s, 1H), 7.31 (s, 7H), 6.92 (s, 1H), 4.59 (d, J=5.9 Hz, 2H); ¹³C NMR (101 MHz, CDCl₃) δ 178.00, 157.25, 152.31, 151.30, 137.30, 128.92, 128.15, 127.96, 122.30, 115.76, 43.57.

### Example 5: Preparation of 5-formyl-N-propylfuran-2-carboxamide

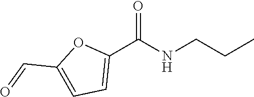

A target compound was obtained (20 mg, 16%) by performing a method similar to that of Example 4.

¹H NMR (400 MHz, CDCl₃) δ 9.67 (s, 1H), 7.27 (d, J=3.7 Hz, 1H), 7.21 (d, J=3.7 Hz, 1H), 6.76 (s, 1H), 3.39 (dt, J=7.2, 6.3 Hz, 1H), 1.62 (tq, J=7.2 Hz, 2H), 0.96 (t, J=7.2 Hz, 2H); ¹³C NMR (101 MHz, CDCl₃) δ 178.08, 157.44, 152.18, 151.67, 122.62, 115.35, 41.25, 22.84, 11.43.

### Example 6: Preparation of 5-formyl-N-methylfuran-2-carboxamide

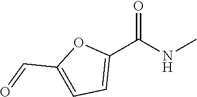

A target compound was obtained (20 mg, 16%) by performing a method similar to that of Example 4.

¹H NMR (400 MHz, CDCl₃) δ 9.69 (s, 1H), 7.28 (d, J=3.7 Hz, 1H), 7.23 (d, J=3.7 Hz, 1H), 6.66 (s, 1H), 3.01 (d, J=5.0 Hz, 3H); ¹³C NMR (101 MHz, CDCl₃) δ 177.97, 158.01, 152.23, 151.53, 122.42, 115.28, 26.13.

### Example 7: Preparation of Benzyl 5-formylfuran-2-carboxylate

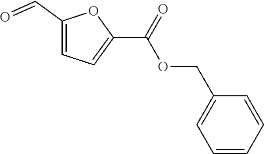 [SMILES: O=Cc1ccc(C(=O)OCc2ccccc2)o1]

A target compound was obtained (9.1 mg, 5.5%) by performing a method similar to that of Example 4.

¹H NMR (400 MHz, CDCl₃) δ 9.81 (s, 1H), 7.47-7.35 (m, 5H), 7.29 (d, J=3.6 Hz, 1H), 7.26 (d, J=3.6 Hz, 1H), 5.39 (s, 2H); ¹³C NMR (101 MHz, CDCl₃) δ 179.14, 157.92, 154.07, 147.71, 135.03, 128.79, 128.68, 127.06, 118.97, 118.74, 67.50.

### Example 8: Preparation of allyl 5-formylfuran-2-carboxylate

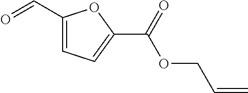

A target compound was obtained (115 mg, 16%) by performing a method similar to that of Example 4.

¹H NMR (400 MHz, CDCl₃) δ 9.80 (s, 1H), 7.28 (d, J=3.6 Hz, 1H), 7.26 (d, J=3.6 Hz, 1H), 6.00 (ddt, J=17.2, 10.4, 5.9 Hz, 1H), 5.41 (dtd, J=17.2, 1.2, 0.6 Hz, 1H), 5.31 (dtd, J=10.4, 1.2, 0.6 Hz, 1H), 4.83 (dt, J=5.9, 1.2 Hz, 2H); ¹³C NMR (101 MHz, CDCl₃) δ 179.11, 157.73, 154.02, 147.70, 131.29, 119.59, 118.85, 118.81, 66.36.

### Example 9: Preparation of 5-(dimethoxymethyl)furan-2-carboxylate

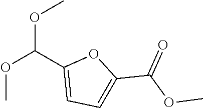

A target compound was obtained (78.3 mg, 51.2%) by performing a method similar to that of Example 4.

¹H NMR (400 MHz, CDCl₃) δ 7.15 (d, J=3.5 Hz, 1H), 6.53 (dd, J=3.5, 0.8 Hz, 1H), 5.45 (s, 1H), 3.88 (s, 3H), 3.36 (s, 6H); ¹³C NMR (101 MHz, CDCl₃) δ 159.10, 155.13, 144.40, 118.48, 110.46, 97.62, 53.16, 52.01.

### Example 10: Preparation of methyl 5-formylfuran-2-carboxylate

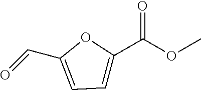

5-Formylfuran-2-carboxylic acid (100 mg, 0.71 mmol) was dissolved in MeOH (1 ml), to which H₂SO₄ (0.1 ml, 1.785 mmol) was added. The reaction mixture was stirred overnight at 60° C. and then dried by evaporation. The reactant was diluted in DCM, and the organic layer was washed with water. The organic layer was dried over Na₂SO₄ and dried by evaporation. The residue was purified by silica gel chromatography (ethyl acetate:hexane=1:3) to give a target compound (88.4 mg, 80.3%).

¹H NMR (400 MHz, CDCl₃) δ 9.80 (s, 1H), 7.26 (s, 2H), 3.95 (s, 3H); ¹³C NMR (101 MHz, CDCl₃) δ 179.02, 158.46, 153.93, 147.69, 118.97, 118.73, 52.64.

### Example 11: Preparation of ethyl 5-(hydroxymethyl)furan-2-carboxylate

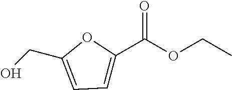

Methyl 5-formylfuran-2-carboxylate (50 mg, 0.32 mmol) was dissolved in EtOH (1 ml), to which NaBH₄ (13.5 mg, 0.36 mmol) was added. The reaction mixture was stirred at room temperature for 2 hours. The reactant was diluted in ethyl acetate, and the organic layer was washed with water. The organic layer was dried over Na₂SO₄ and dried by evaporation. The residue was purified by silica gel chromatography (ethyl acetate:hexane=1:1) to give a target compound (48 mg, 88%).

¹H NMR (400 MHz, CDCl₃) δ 7.10 (d, J=3.4 Hz, 1H), 6.39 (d, J=3.4 Hz, 1H), 4.66 (s, 2H), 4.34 (q, J=7.1 Hz, 2H), 1.35 (t, J=7.1 Hz, 3H); ¹³C NMR (101 MHz, CDCl₃) δ 158.95, 158.41, 144.37, 118.74, 109.44, 61.11, 57.58, 14.38.

### Example 12: Preparation of Furan-2,5-diyldimethanol

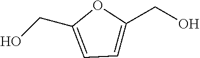

5-Hydroxylfurfural (50 mg, 0.4 mmol) was dissolved in distilled water (0.8 ml). NaBH₄ (16.5 mg, 0.44 mmol in 0.3 ml water) was added dropwise to the well stirred solution. The reaction mixture was stirred at room temperature for 2 hours. The reactant was diluted in ethyl acetate, and the organic layer was washed with water. The organic layer was dried over Na₂SO₄ and dried by evaporation to give a target compound (40.8 mg, 80%).

¹H NMR (400 MHz, CDCl₃) δ 6.24 (s, 2H), 4.60 (s, 4H), 1.75 (s, 2H); ¹³C NMR, (101 MHz, CDCl₃) δ 154.14, 108.66, 57.65.

### Example 13: Preparation of methyl 5-(hydroxymethyl)furan-2-carboxylate

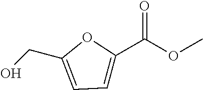

A target compound was obtained (20.3 mg, 99%) by performing a method similar to that of Example 11.

¹H NMR (400 MHz, CDCl₃) δ 7.11 (d, J=3.4 Hz, 1H), 6.39 (d, J=3.4 Hz, 1H), 4.65 (s, 2H), 3.87 (s, 3H), 2.54 (s, 1H); ¹³C NMR (101 MHz, CDCl₃) δ 159.29, 158.54, 144.05, 118.98, 109.49, 57.56, 52.03.

### Example 14: Preparation of 5-(hydroxymethyl)furan-2-carboxylic Acid

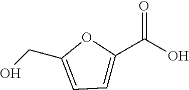

Ethyl 5-(hydroxymethyl)furan-2-carboxylate (20.7 mg, 0.122 mmol) was dissolved in distilled water (0.4 ml), to which NaOH (4.87 mg, 0.122 mmol) was added. The reaction mixture was stirred for 2 hours. The reactant was diluted in ethyl acetate, and the organic layer was washed with water and 1 M HCl solution. The organic layer was dried over Na₂SO₄ and dried by evaporation to give a target compound (15.7 mg, 92%) without further purification.

¹H NMR (400 MHz, CDCl₃) δ 7.16 (d, J=3.4 Hz, 1H), 6.47 (d, J=3.4 Hz, 1H), 4.60 (s, 2H), 4.51 (s, 1H); ¹³C NMR (101 MHz, CDCl₃) δ 160.08, 158.69, 144.11, 118.75, 108.79, 56.59.

### Example 15: Preparation of 1-(5-(((tetrahydro-2H-pyran-2-yl)oxy)methyl)furan-2-yl)ethanol

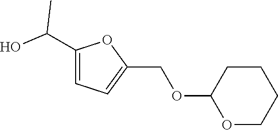 [SMILES: CC(O)c1ccc(COC2CCCCO2)o1]

5-(((Tetrahydro-2H-pyran-2-yl)oxy)methyl)furan-2-carbaldehyde (669 mg, 3.2 mmol) was dissolved in Et₂O under inert conditions. The mixture was cooled to −30° C. and 3 M CH₃MgBr (1.07 ml, 3.2 mmol in Et₂O) was added dropwise thereto. The reactant was stirred at room temperature for 2 hours and quenched with saturated NH₄Cl. The reaction mixture was diluted in Et₂O and the organic layer was washed with water and brine. The organic layer was dried over Na₂SO₄ and dried by evaporation. The residue was purified by silica gel chromatography (ethyl acetate:hexane=1:4) to give a target compound (238.5 mg, 33%).

¹H NMR (400 MHz, CDCl₃) δ 6.24 (d, J=3.2 Hz, 1H), 6.16 (d, 1H), 4.84 (q, J=6.6 Hz, 1H), 4.70 (t, J=4.3 Hz, 1H), 4.62 (d, J=12.9 Hz, 1H), 4.45 (d, J=12.9 Hz, 1H), 2.22 (s, 1H), 1.88-1.42 (m, 6H), 1.51 (d, J=6.6 Hz, 3H); ¹³C NMR (101 MHz, CDCl₃) δ 158.11, 151.11, 110.04, 105.81, 97.39, 63.64, 62.09, 60.80, 30.40, 25.46, 21.22, 19.23.

### Example 16: Preparation of 1-(5-(hydroxymethyl)furan-2-yl)ethanol

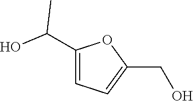

1-(5-(((Tetrahydro-2H-pyran-2-yl)oxy)methyl)furan-2-yl)ethanol (50 mg, 0.225 mmol) and p-TsOH (4 mg, 0.0225 mmol) were dissolved in distilled water. The reaction mixture was stirred for 4 hours, diluted in ethyl acetate, and the organic layer was washed with water and brine. The organic layer was dried over Na₂SO₄ and dried by evaporation. The residue was purified by silica gel chromatography (ethyl acetate:hexane=2:1) to give a target compound (18.22 mg, 57%).

¹H NMR (400 MHz, CDCl₃) δ 6.20 (d, J=3.2 Hz, 1H), 6.16 (d, J=3.2 Hz, 1H), 4.84 (q, J=6.6 Hz, 1H), 4.56 (s, 2H), 2.37 (s, 1H), 1.52 (d, J=6.6 Hz, 3H); ¹³C NMR (101 MHz, CDCl₃) δ 157.72, 153.48, 108.42, 105.95, 63.64, 57.50, 21.21.

### Example 17: Preparation of Furan-2,5-dicarbaldehyde

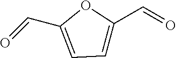

5-Hydroxylfurfural (200 mg, 1.59 mmol) was dissolved in DCM (20 ml). PCC (513 mg, 2.38 mmol) and celite (400 mg) were added to the solution. The mixture was stirred for 2 hours and then filtered through celite. The filtrate was dried by evaporation. The residue was purified by silica gel chromatography (ethyl acetate:hexane=1:1) to give a target compound (146 mg, 74%).

¹H NMR (400 MHz, CDCl₃) δ 9.84 (s, 2H), 7.33 (s, 2H); ¹³C NMR (101 MHz, CDCl₃) δ 179.27, 154.29, 119.36.

### Example 18: Preparation of 5-(1-hydroxyethyl)furan-2-carbaldehyde

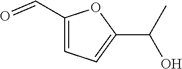

Furan-2,5-dicarbaldehyde (98 mg, 0.79 mmol) was dissolved in THF (4 ml) under inert conditions. The mixture was cooled to −20° C. and 3 M CH₃MgBr (0.26 ml, 0.79 ml in Et₂O) was added dropwise thereto. The reactant was stirred at −20° C. for 1 hour and quenched with saturated NH₄Cl. The reaction mixture was diluted in ethyl acetate and the organic layer was washed with water and brine. The organic layer was dried over Na₂SO₄ and dried by evaporation. The residue was purified by silica gel chromatography (ethyl acetate:hexane=1:1) to give a target compound (11 mg, 10%).

¹H NMR (400 MHz, CDCl₃) δ 9.57 (s, 1H), 7.19 (d, J=3.6 Hz, 1H), 6.46 (d, J=3.6 Hz, 1H), 4.95 (q, J=6.4 Hz, 1H), 2.61 (s, 1H), 1.58 (d, J=6.4 Hz, 3H); ¹³C NMR (101 MHz, CDCl₃) δ 177.72, 164.49, 152.12, 122.81, 108.02, 63.96, 21.56.

### Example 19: Preparation of 1-(5-formylfuran-2-yl)ethyl Acetate

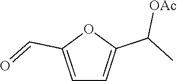

5-(1-Hydroxypropyl)furan-2-carbaldehyde (50 mg, 0.36 mmol) was dissolved in DCM (1 ml), to which TEA (0.05 ml, 0.36 mmol) was added. The mixture was cooled to 0° C. and acetyl chloride (0.026 ml, 0.36 mmol) was added dropwise thereto. After adjusting the temperature of the mixture to room temperature, the mixture was stirred for 4 hours. The solution was quenched with 5% aqueous HCl. The organic layer was washed with water and brine, and dried over Na₂SO₄. The residue was purified by silica gel chromatography (ethyl acetate:hexane=1:2) to give a target compound (28 mg, 43%).

¹H NMR (400 MHz) δ 9.61 (s, 1H), 7.18 (d, J=3.6 Hz, 1H), 6.49 (d, J=3.6 Hz, 1H), 5.95 (q, J=6.7 Hz, 1H), 2.07 (s, 3H), 1.61 (d, J=6.8 Hz, 4H).

### Example 20: Preparation of 5-(1-hydroxypropyl)furan-2-carbaldehyde

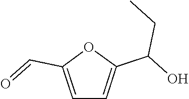

Furan-2,5-dicarbaldehyde (100 mg, 0.81 mmol) was dissolved in THF (1 ml) under inert conditions. The mixture was cooled to −20° C., and 1 M C₂H₅MgBr (0.97 ml, 0.97 mmol, dissolved in THF) was added dropwise thereto. The reaction mixture was stirred at the same temperature for 1 hour and quenched with saturated NH₄Cl. The reaction mixture was diluted in ethyl acetate and the organic layer was washed with water and brine. The organic layer was dried over Na₂SO₄. The residue was purified by silica gel chromatography (ethyl acetate:hexane=1:1.5) to give a target compound (15 mg, 12%).

¹H NMR (400 MHz) δ 9.58 (s, 1H), 7.20 (d, J=3.5 Hz, 1H), 6.47 (d, J=3.5 Hz, 1H), 4.71 (dd, J=7.2, 5.8 Hz, 1H), 2.00-1.80 (m, 2H), 0.98 (t, J=7.4 Hz, 3H); ¹³C NMR (101 MHz) δ 177.60, 163.61, 152.23, 122.56, 108.68, 69.41, 28.90, 9.61.

### Example 21: Preparation of 1-(5-formylfuran-2-yl)propyl Acetate

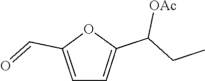

5-(1-Hydroxypropyl)furan-2-carbaldehyde (11 mg, 0.07 mmol) was dissolved in DCM (0.2 ml), to which TEA (0.01 ml, 0.08 mmol) was added. The reaction mixture was cooled to 0° C. and acetyl chloride (0.006 ml, 0.08 mmol) was added dropwise thereto. After adjusting the temperature of the reaction mixture to room temperature, the mixture was stirred for 4 hours. The reaction mixture was quenched with 5% aqueous HCl. The organic layer was washed with water and brine, and dried over Na₂SO₄. The residue was purified by silica gel chromatography (ethyl acetate:hexane=1:2) to give a target compound 6.2 mg, 45%).

¹H NMR (400 MHz) δ 9.62 (s, 1H), 7.19 (d, J=3.6 Hz, 1H), 6.49 (d, J=3.5 Hz, 1H), 5.79 (t, J=7.0 Hz, 1H), 2.10 (s, 3H), 2.06-1.96 (m, 2H), 0.93 (t, J=7.4 Hz, 3H); ¹³C NMR (101 MHz) δ 177.81, 170.17, 159.00, 152.52, 121.60, 110.78, 69.99, 25.98, 20.99, 9.58.

<Evaluation of Pharmacological Effects of Compounds>

In order to evaluate the therapeutic effect of the compound represented by formula 1 according to the present invention on poliosis or vitiligo and the preventing effect on gray hair, the following pharmacological effect evaluation experiments were performed. Particularly, cell viability, cell differentiation, intracellular melanin content and cell migration of melanoblasts according to the treatment of the compounds of Examples of the present invention were evaluated.

<Culture of Melanoblast Cell Line>

First, a melanoblast cell line for use in the experiment was cultured.

Particularly, Melb-a (melanoblast) was purchased from Functional Genomics Cell Bank (London, UK), Welcome Trust. Melb-a was cultured in a 37° C., 10% CO₂ incubator, and subcultured in a medium in which 20 nM PDBu (Phorbol12,13-dibutyrate; Sigma Chemical Co., St Louis, USA), 1 ng/ml bFGF (Murine FGF (fibroblast growth factor)-basic; PeproTech), 5% fetal bovine serum (Invitrogen Corp., CA, USA) and 2 nM L-glutamine were added to RPMI 1640 supplemented with 1% pecicilline (Invitrogen Corp, CA, USA)/streptomycin (Invitrogen Corp, CA, USA) to promote growth.

After treating the cells with trypsin-EDTA (Invitrogen Corp., CA, USA), the cells were precipitated and suspended in complete RPMI 1640 medium (Invitrogen Corp., CA, USA). The number of viable cells was measured using the trypan blue exclusion method and a hemocytometer. Complete RPMI 1640 medium was placed in 10 cm cell culture dishes, and the cells were inoculated at the concentration of 2×10⁵ cells/dish.

### Experimental Example 1: Confirmation of Melanoblast Cell Viability

### <1-1> Confirmation of Cell Viability According to Treatment Concentration of Compound of Example 1

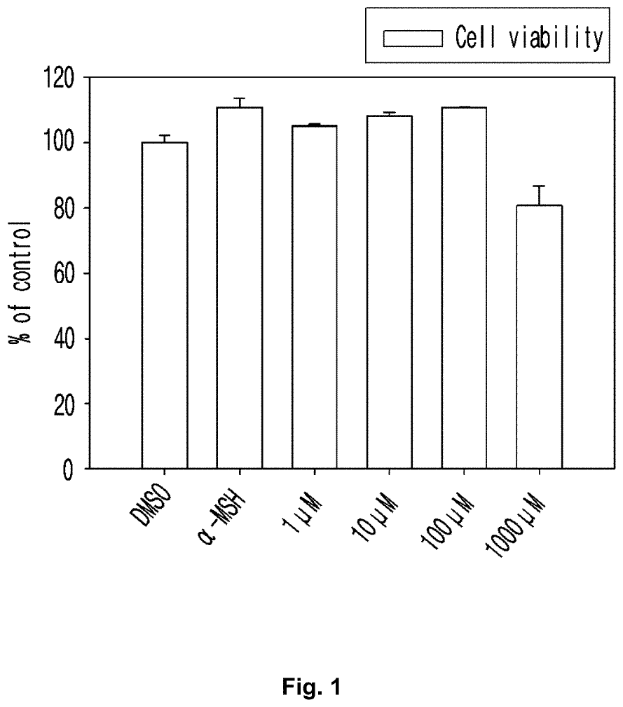

The following experiment was performed to confirm the melanoblast cell viability according to the treatment concentration of the compound of Example 1, and the results are shown in FIG. 1 and Table 1.

Particularly, Melb-a cells (4×10³ cells/well) were placed in a 96 well plate (200 μl/well), and pre-cultured in a 37° C., 10% CO₂ incubator for 24 hours. After adding a culture solution containing the example compound at the concentration of 1 μM, 10 μM, 100 μM, or 1000 μM to the adhered cells, the culture solution was exchanged every two days for 4 days of culture (samples were treated twice). DMSO (negative control) and α-MSH (α-Melanocyte-stimulating hormone, positive control)) were treated with 0.8 μM. After 96 hours, formazan formed by treating MTT (3-(4,5-dimethylthiazol-2-yl)-2,5-diphenyltetrazoliumbromide, Sigma Chemical Co., St Louis, USA) at the concentration of 5 mg/ml was dissolved by DMSO and OD₅₄₀ was measured with an ELISA microplate reader. The results are shown in FIG. 1 and Table 1.

TABLE 1

| | Cell viability |
|---|---|
| | (%) |
| Negative control (DMSO) | 100.00 ± 2.11 |
| Positive control (α-MSH) | 110.71 ± 2.88 |
| 1 μM | 105.08 ± 0.52 |
| 10 μM | 108.03 ± 1.36 |
| 100 μM | 110.71 ± 0.17 |
| 1000 μM | 80.70 ± 6.02 |

As shown in FIG. 1 and Table 1, when the compound of Example 1 according to the present invention was treated at the concentrations of 1-100 μM, the cell viability was 105% or more, indicating that the compound did not exhibit toxicity to melanoblasts.

<1-2> Confirmation of Cell Viability According to Treatment of Compounds of Examples 2-10 at 10 μM (MTT Assay)

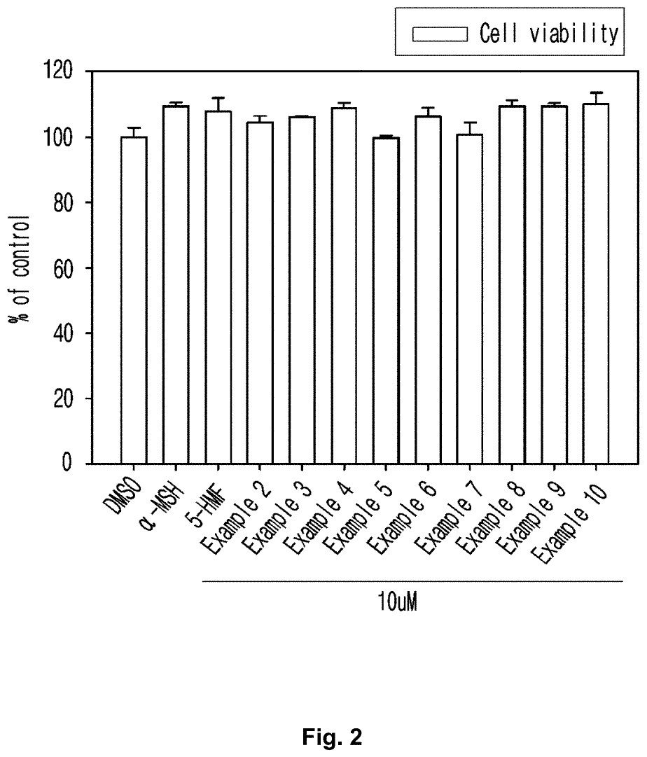

In order to confirm the cell viability of melanoblasts when the compounds of Examples 2-10 were treated at the concentration of 10 μM, an experiment was performed in the same manner as described in Experimental Example <1-1>, and the results are shown in FIG. 2 and Table 2.

TABLE 2

| | Cell viability |
|---|---|
| | (%) |
| Negative control (DMSO) | 100.00 ± 2.83 |
| Positive control (α-MSH) | 109.46 ± 1.08 |
| 5HMF 10 μM | 107.92 ± 3.98 |
| Example 2 | 104.38 ± 2.10 |
| Example 3 | 106.14 ± 0.48 |
| Example 4 | 108.85 ± 1.53 |
| Example 5 | 99.69 ± 0.79 |
| Example 6 | 106.34 ± 2.59 |
| Example 7 | 100.68 ± 3.60 |
| Example 8 | 109.41 ± 1.93 |
| Example 9 | 109.42 ± 0.89 |
| Example 10 | 110.12 ± 3.33 |

As shown in FIG. 2 and Table 2, when the compounds of Examples 2-10 according to the present invention were treated at the concentration of 10 μM, the cell viability was 99% or more, indicating that the compounds did not exhibit toxicity to melanoblasts.

<1-3> Confirmation of Cell Viability According to Treatment Concentration of Compounds of Examples 9 and 10

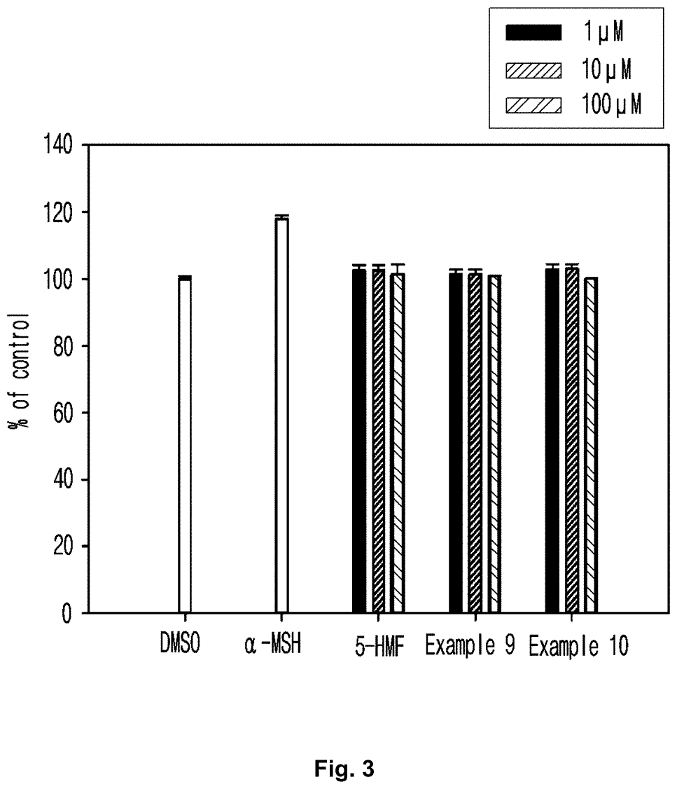

In order to confirm the cell viability of melanoblasts according to the treatment concentration of the compounds of Examples 9 and 10, an experiment was performed in the same manner as described in Experimental Example <1-1>, and the results are shown in FIG. 3. As shown in FIG. 3, when the compounds of Examples 9 and 10 according to the present invention were treated at the concentrations of 1-100 μM, the cell viability was 99% or more, indicating that the compounds did not exhibit toxicity to melanoblasts.

<1-4> Confirmation of Cell Viability According to Treatment of Compounds of Examples 11-18 at 10 μM

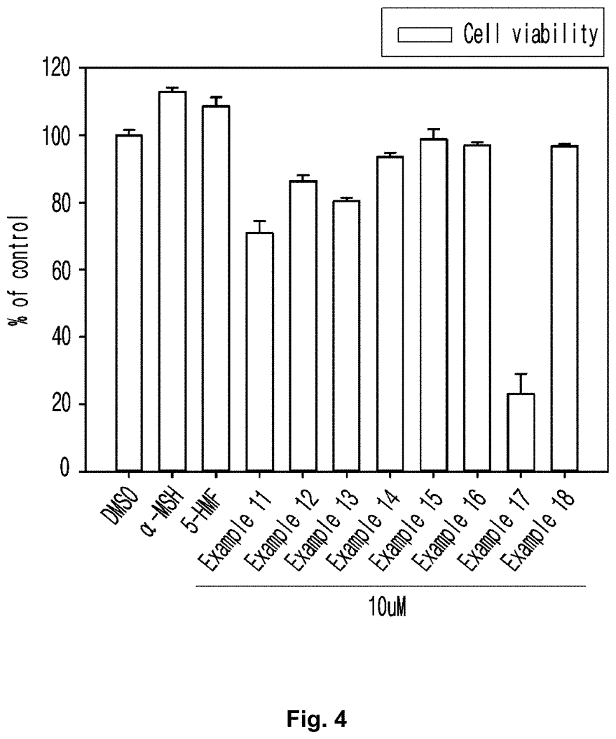

In order to confirm the cell viability of melanoblasts when the compounds of Examples 11-18 were treated at the concentration of 10 μM, an experiment was performed in the same manner as described in Experimental Example <1-1>, and the results are shown in FIG. 4 and Table 3.

TABLE 3

| | Cell viability (%) |
|---|---|
| Negative control (DMSO) | 100.00 ± 1.60 |
| Positive control (α-MSH) | 112.79 ± 1.47 |
| Example 1 10 μM | 108.42 ± 2.76 |
| Example 11 | 70.96 ± 3.49 |
| Example 12 | 86.31 ± 1.78 |
| Example 13 | 80.38 ± 1.03 |
| Example 14 | 93.49 ± 1.26 |
| Example 15 | 98.71 ± 3.07 |
| Example 16 | 97.02 ± 0.93 |
| Example 17 | 23.08 ± 5.95 |
| Example 18 | 96.69 ± 0.62 |

As shown in FIG. 4 and Table 3, when the compounds of Examples 11-18 according to the present invention were treated at the concentration of 10 μM, the cell viability was low compared to when the compound of Example 1 was treated, but when the compounds of Examples 12-16 and 18 were treated, the cell viability was 80% or more, indicating a safe level of cytotoxicity. When the compound of Example 11 was treated, the cell viability was 70%, which was high in cytotoxicity, and when the compound of Example 17 was treated, the cell viability was very low.

<1-5> Confirmation of Cell Viability According to Treatment Concentration of Compound of Example 18

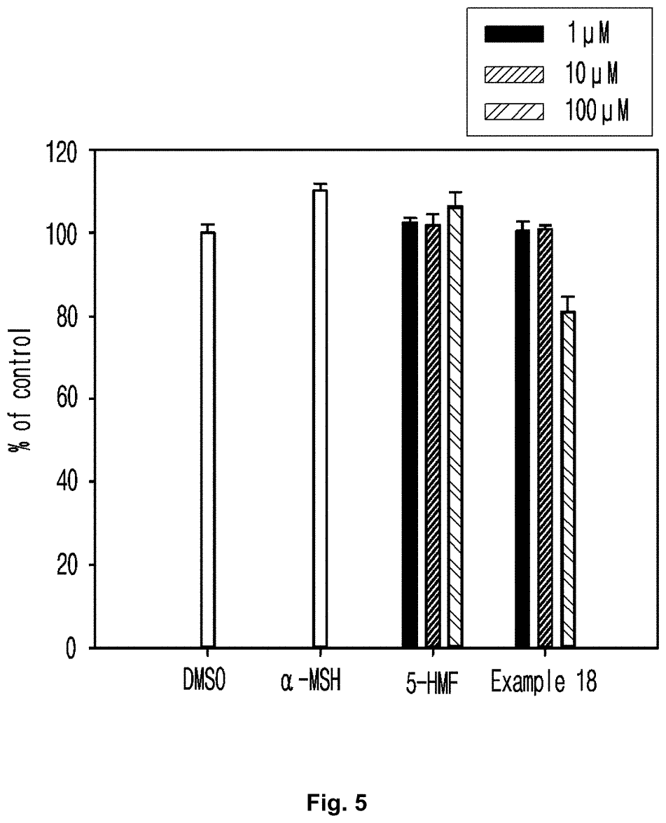

In order to confirm the cell viability of melanoblasts according to the treatment concentration of the compound of Example 18, an experiment was performed in the same manner as described above, and the results are shown in FIG. 5. As shown in FIG. 5, when the compound of Example 18 according to the present invention was treated at the concentrations of 1-100 μM, the cell viability was more than 80%, and when treated at the concentrations of 1-10 μM, the cell viability was more than 100%, similar to that when the compound of Example 1 was treated, indicating that the compound did not exhibit toxicity to melanoblasts.

<1-6> Confirmation of Cell Viability According to Treatment of Compounds of Examples 19-21 at 10 μM

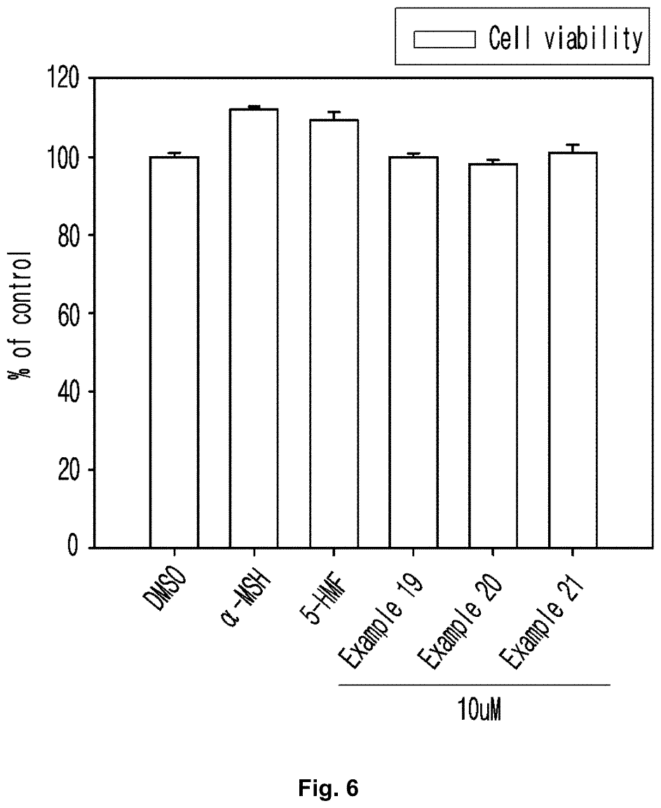

In order to confirm the cell viability of melanoblasts when the compounds of Examples 11-18 were treated at the concentration of 10 μM, an experiment was performed in the same manner as described in Experimental Example <1-1>, and the results are shown in FIG. 6 and Table 4.

TABLE 4

| | Cell viability (%) |
|---|---|
| Negative control (DMSO) | 100.00 ± 0.97 |
| Positive control (α-MSH) | 112.15 ± 0.64 |
| Example 1 | 109.48 ± 1.95 |
| Example 19 | 99.91 ± 1.03 |
| Example 20 | 98.04 ± 1.16 |
| Example 21 | 101.15 ± 1.90 |

As shown in FIG. 6 and Table 4, when the compounds of Examples 19-21 according to the present invention were treated at the concentration of 10 μM, the cell viability was 98% or more, indicating a safe level of cytotoxicity.

Therefore, the compound represented by formula 1 according to the present invention did not show toxicity to melanoblasts associated with the production of melanin, and thus it can be effectively used for the prevention, alleviation or treatment of poliosis or vitiligo.

### Experimental Example 2: Evaluation of Intracellular Melanin Content (Melanin Assay)

Melanoblasts are non-pigmented cells in which the tyrosinase function is stopped, and the melanoblast differentiation can be measured using the melanin content. Accordingly, the following experiment was performed to confirm the melanoblast differentiation and intracellular melanin content according to the treatment of the Example compound of the present invention.

Particularly, melanoblasts were cultured in the same manner as in the cell line culture of Experimental Example 1, and after 96 hours of culture, the culture medium was removed. 1 ml of PBS was added, washed twice, and then cells were collected by adding trypsin-EDTA. After washing the cells twice by adding 1 ml of PBS (phosphate buffered saline, Invitrogen Corp., CA, USA) per well, trypsin-EDTA was added to harvest the cells. The harvested cells were transferred to a 1.5 ml Eppendorf tube, and a cell pellet was obtained by centrifugation. 100 μl of 1N NaOH solution containing 10% DMSO was put into each of the cells thus collected, suspended, and reacted at 80° C. for 1 hour. 1 N NaOH solution containing 10% DMSO was added to the collected cells (100 μl/well). The cells were suspended and reacted at 80° C. for 1 hour. The reacted and dissolved melanin in cells was measured in comparison with the control group at 405 nm using a plate reader.

<2-1> Confirmation of Melanin Content According to Treatment Concentration of Compound of Example 1

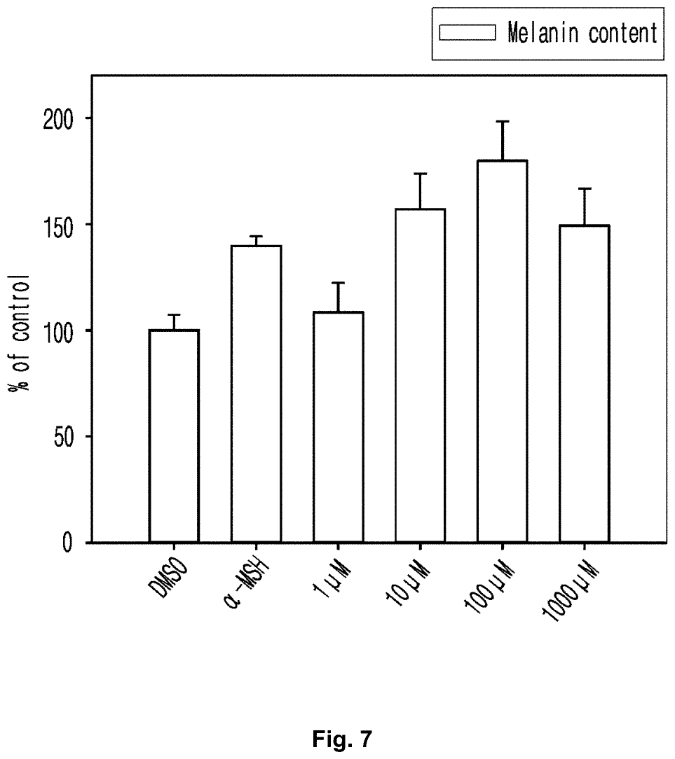

The melanin content in melanoblasts according to the treatment concentration of the compound of Example 1 was confirmed, and the results are shown in FIG. 7 and Table 5.

TABLE 5

| | Melanin content compared |
|---|---|
| | to negative control (%) |
| Negative control (DMSO) | 100.00 ± 7.48 |
| Positive control (α-MSH) | 139.82 ± 4.53 |
| 1 μM | 108.54 ± 13.90 |
| 10 μM | 157.04 ± 16.68 |
| 100 μM | 179.77 ± 18.58 |
| 1000 μM | 149.37 ± 17.44 |

As shown in FIG. 7 and Table 5, when the compound of Example 1 according to the present invention was treated at the concentrations of 1-100 μM, the melanin content was gradually increased compared to the negative control, and when treated at the concentration of 1000 μM, the melanin content was rather reduced. From the above results, it was confirmed that the compound of Example 1 according to the present invention promoted and increased the differentiation of melanoblasts.

<2-2> Confirmation of Melanin Content According to Treatment of Compounds of Examples 2-10 at 10 μM

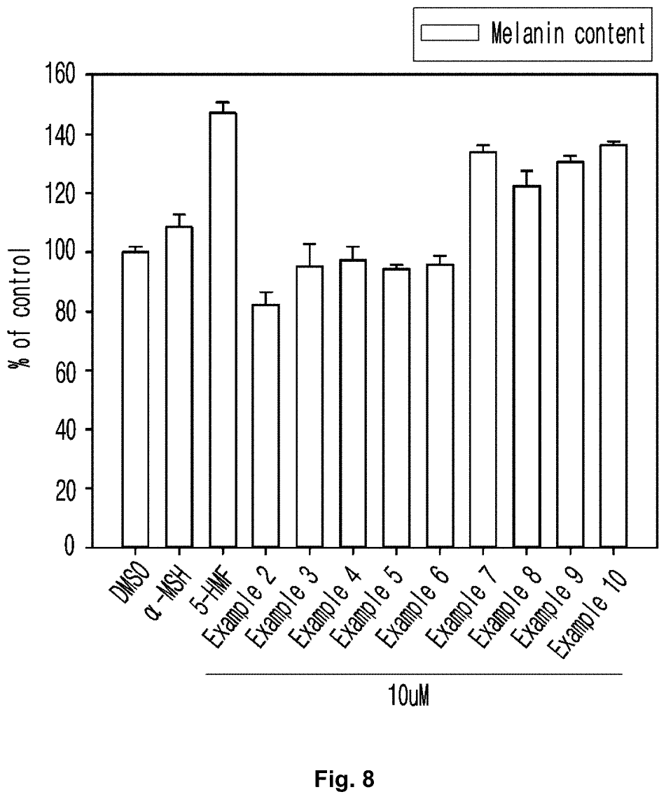

The melanin content in melanoblasts according to the treatment of the compounds of Examples 2-10 at the concentration of 10 μM was confirmed, and the results are shown in FIG. 8 and Table 6.

TABLE 6

| | Melanin content compared |
|---|---|
| | to negative control (%) |
| Negative control (DMSO) | 100.00 ± 1.87 |
| Positive control (α-MSH) | 108.39 ± 4.30 |
| Example 1 10 μM | 147.05 ± 3.73 |
| Example 2 | 82.36 ± 4.25 |
| Example 3 | 95.24 ± 7.54 |
| Example 4 | 97.40 ± 4.47 |
| Example 5 | 94.29 ± 1.51 |
| Example 6 | 95.94 ± 2.87 |
| Example 7 | 133.86 ± 2.32 |
| Example 8 | 122.44 ± 5.15 |
| Example 9 | 130.67 ± 2.03 |
| Example 10 | 136.26 ± 1.19 |

As shown in FIG. 8 and Table 6, when the compounds of Examples 2-6 according to the present invention were treated at the concentration of 10 μM, the melanin content was lowered compared to when the negative control was treated, but when the compounds of Examples 7-10 were treated, the melanin content was significantly increased compared to when the negative control was treated.

<2-3> Confirmation of Melanin Content According to Treatment Concentration of Compounds of Examples 9 and 10

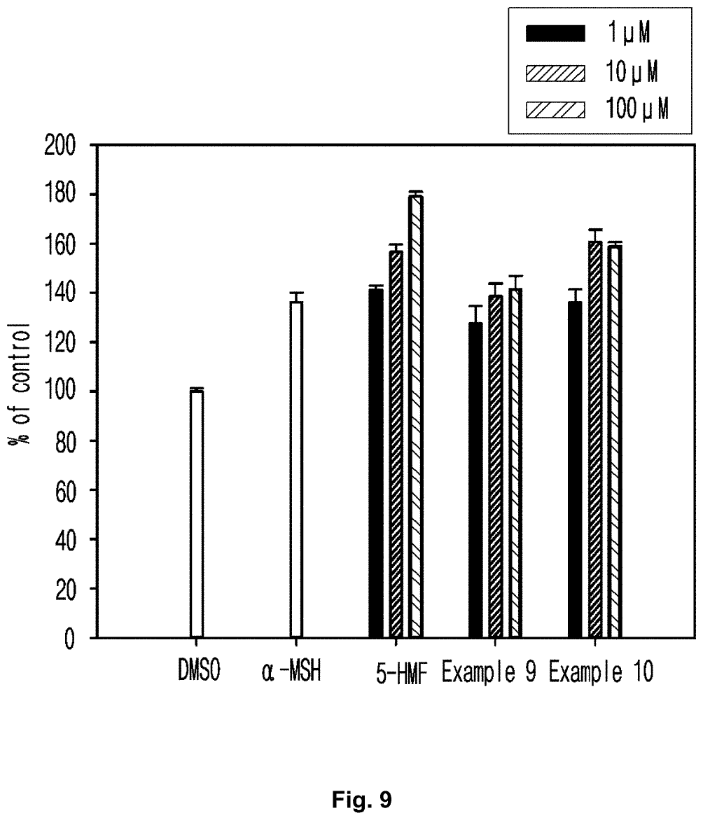

The melanin content in melanoblasts according to the treatment concentration of the compounds of Examples 9 and 10 was confirmed, and the results are shown in FIG. 9 and Table 7.

TABLE 7

| | Melanin content compared |
|---|---|
| | to negative control (%) |
| Negative control (DMSO) | 100.00 ± 1.36 |
| Positive control (α-MSH) | 136.46 ± 3.74 |
| Example 1 1 μM | 141.34 ± 1.98 |
| Example 1 10 μM | 156.63 ± 2.95 |
| Example 1 100 μM | 179.37 ± 1.77 |
| Example 9 1 μM | 127.72 ± 7.18 |
| Example 9 10 μM | 138.86 ± 5.18 |
| Example 9 100 μM | 141.62 ± 5.43 |
| Example 10 1 μM | 136.37 ± 5.19 |
| Example 10 10 μM | 160.87 ± 4.87 |
| Example 10 100 μM | 158.93 ± 1.77 |

As shown in FIG. 9 and Table 7, when the compound of Example 9 according to the present invention was treated at the concentrations of 1-100 μM, the melanin content was gradually increased compared to the negative control, and when the compound of Example 10 was treated at the concentrations of 1-100 μM, the melanin content was gradually increased compared to the negative control. When the compound of Example 10 was treated at the concentration of 100 μM, the melanin content was somewhat reduced.

<2-4> Confirmation of Melanin Content According to Treatment of Compounds of Examples 11-18 at 10 μM

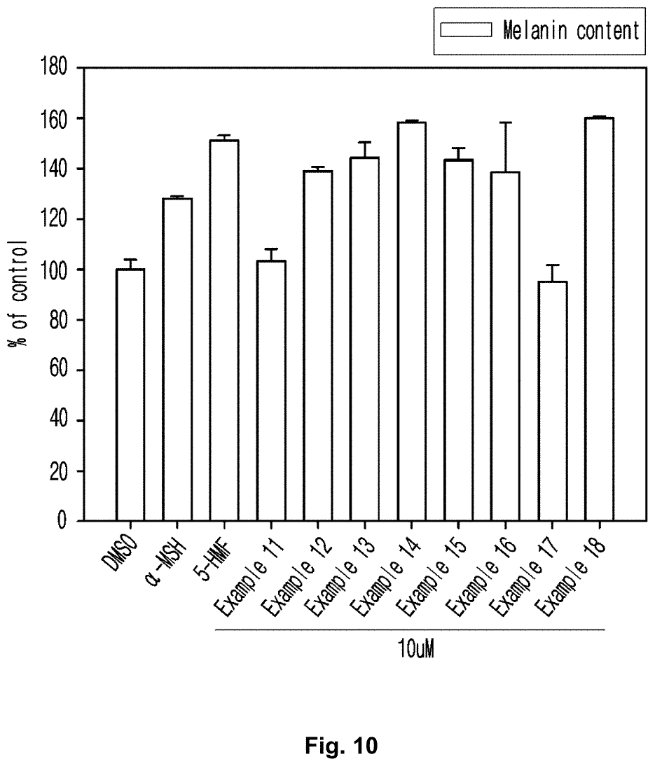

The melanin content in melanoblasts according to the treatment of the compounds of Examples 11-18 at the concentration of 10 μM was confirmed, and the results are shown in FIG. 10 and Table 8.

TABLE 8

| | Melanin content compared |
|---|---|
| | to negative control (%) |
| Negative control (DMSO) | 100.00 ± 4.04 |
| Positive control (α-MSH) | 128.13 ± 1.11 |
| Example 1 10 μM | 151.10 ± 2.15 |
| Example 11 | 103.59 ± 4.63 |
| Example 12 | 139.12 ± 1.60 |
| Example 13 | 144.56 ± 5.90 |
| Example 14 | 158.45 ± 0.75 |
| Example 15 | 143.58 ± 4.59 |
| Example 16 | 138.60 ± 19.89 |
| Example 17 | 95.31 ± 6.49 |
| Example 18 | 160.19 ± 0.47 |

As shown in FIG. 10 and Table 8, when the compound of Example 17 according to the present invention was treated at the concentration of 10 μM, the melanin content was lowered compared to when the negative control was treated, but when the compounds of Examples 11-16 and 18 were treated, the melanin content was significantly increased compared to when the negative control was treated.

<2-5> Confirmation of Melanin Content According to Treatment Concentration of Compound of Example 18

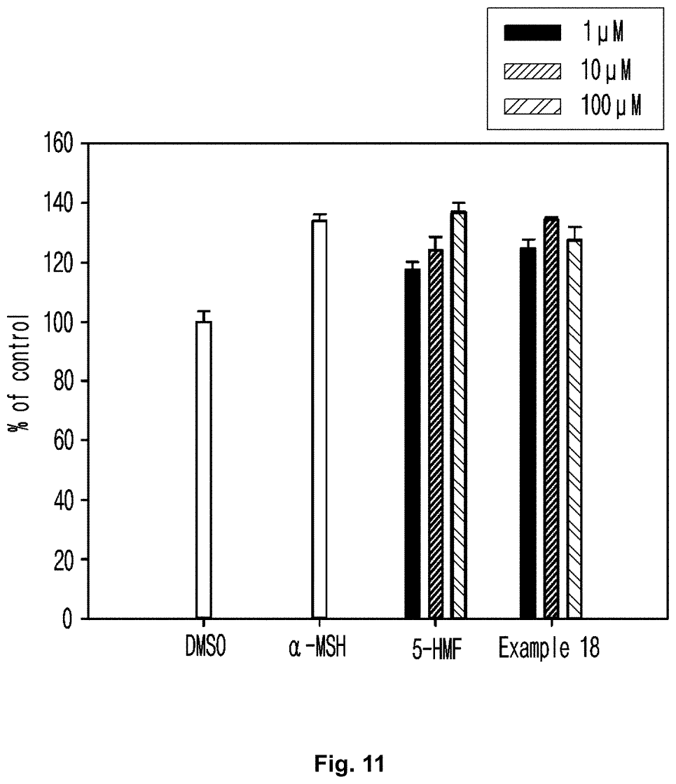

The melanin content in melanoblasts according to the treatment concentration of the compound of Example 18, and the results are shown in FIG. 11 and Table 9.

TABLE 9

| | Melanin content compared |
|---|---|
| | to negative control (%) |
| Negative control (DMSO) | 100.00 ± 3.61 |
| Positive control (α-MSH) | 134.07 ± 2.26 |
| Example 1 1 μM | 117.6 ± 2.59 |
| Example 1 10 μM | 124.07 ± 4.52 |
| Example 1 100 μM | 137.15 ± 3.06 |
| Example 18 1 μM | 124.84 ± 3.04 |
| Example 18 10 μM | 134.57 ± 0.69 |
| Example 18 100 μM | 127.78 ± 4.15 |

As shown in FIG. 11 and Table 9, when the compound of Example 18 according to the present invention was treated at the concentrations of 1-100 μM, the melanin content was slightly increased compared to when the negative control was treated, and when the compound was treated at the concentration of 100 μM, the melanin content was decreased.

<2-6> Confirmation of Melanin Content According to Treatment of Compounds of Examples 19-21 at 10 μM

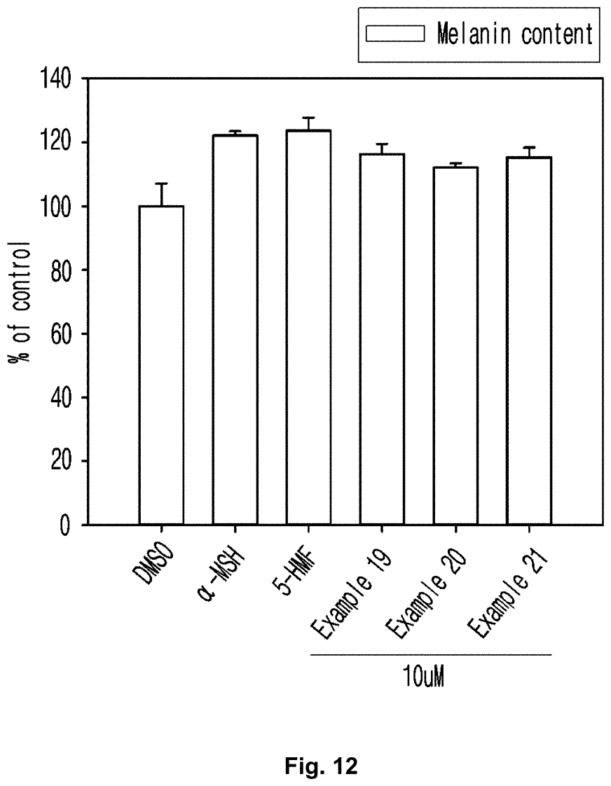

The melanin content in melanoblasts according to the treatment of the compounds of Examples 19-21 at the concentration of 10 μM was confirmed, and the results are shown in FIG. 12 and Table 10.

TABLE 10

| | Melanin content compared |
|---|---|
| | to negative control (%) |
| Negative control (DMSO) | 100.00 ± 6.96 |
| Positive control (α-MSH) | 122.15 ± 1.21 |
| Example 1 | 123.60 ± 4.13 |
| Example 19 | 116.44 ± 3.15 |
| Example 20 | 112.01 ± 1.50 |
| Example 21 | 115.37 ± 2.96 |

As shown in FIG. 12 and Table 10, when the compounds of Examples 19-21 according to the present invention were treated, the melanin content was increased compared to when the negative control was treated.

Therefore, the compound represented by formula 1 according to the present invention promoted and increased the differentiation of melanoblasts to increase the intracellular melanin content, and thus it can be effectively used for the prevention, alleviation or treatment of poliosis or vitiligo.

### Experimental Example 3: Confirmation of Melanoblast Migration Effect

Melanoblasts move to the epidermis and differentiate into melanocytes, which are cells with dendrites, and melanin is formed in the melanocytes. Therefore, in order to increase the content of melanin, the migration of melanoblasts is important, so it is possible to prove the therapeutic effect on poliosis or vitiligo by confirming the migration of melanoblasts. Accordingly, the following experiment was performed to confirm the migration of melanoblasts according to the treatment of the Example compound of the present invention.

<3-1> Confirmation of Cell Migration Effect According to Treatment Concentration of Compound of Example 1

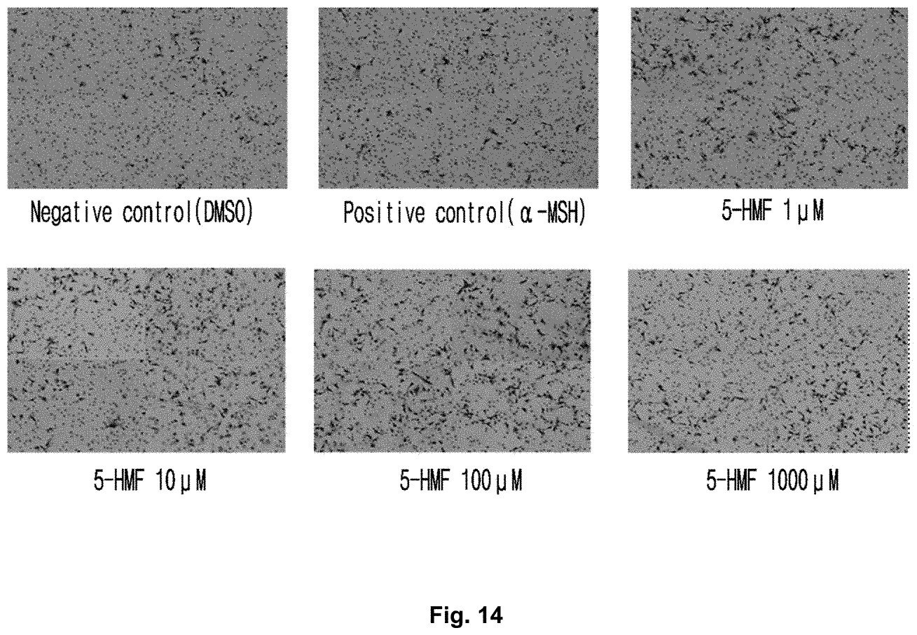

The melanoblast migration effect according to the treatment concentration of the compound of Example 1 was confirmed, and the results are shown in FIG. 14 and Table 11.

Particularly, the melanoblast migration according to the treatment of the compound of Example 1 was confirmed by Transwell migration assay. In detail, the cell migration was measured using a transwell cell culture chamber. A 0.8 μm polyvinylpyrroliodone-free polycarbonate filter in the transwell cell culture chamber was coated with 1% gelatin. After hardening the coated cell insert, 600 μl of serum-free RPMI medium was added to the bottom of the chamber, and 100 μl of melb-a cells (2×10⁶ cells/ml) were inoculated on the chamber, followed by culture for 24 hours. The concentrations of the test samples of the present invention were 1 μM, 10 μM, 100 μM, and 1000 μM, and a-MSH, the positive control, was treated at the concentration of 100 nM. After 24 hours, the filter was cut out and fixed with methanol. After staining with hematoxylin and eosin, the non-migrated cells on the filter were removed by wiping with a cotton swab. The lower part of the filter was observed under a microscope to see the migrated cells. To measure the number of cells, the filter was divided into quarters and observed under a microscope at 40 times magnification, and the average value was obtained. The experiment was repeated three times under each condition.

TABLE 11

| | Cell migration rate (%) |
|---|---|
| Negative control (DMSO) | 100.00 ± 1.21% |
| Positive control (α-MSH) | 135.04 ± 21.48% |
| 1 μM | 201.71 ± 9.59% |
| 10 μM | 276.92 ± 15.71% |
| 100 μM | 394.02 ± 10.74% |
| 1000 μM | 252.99 ± 13.38% |

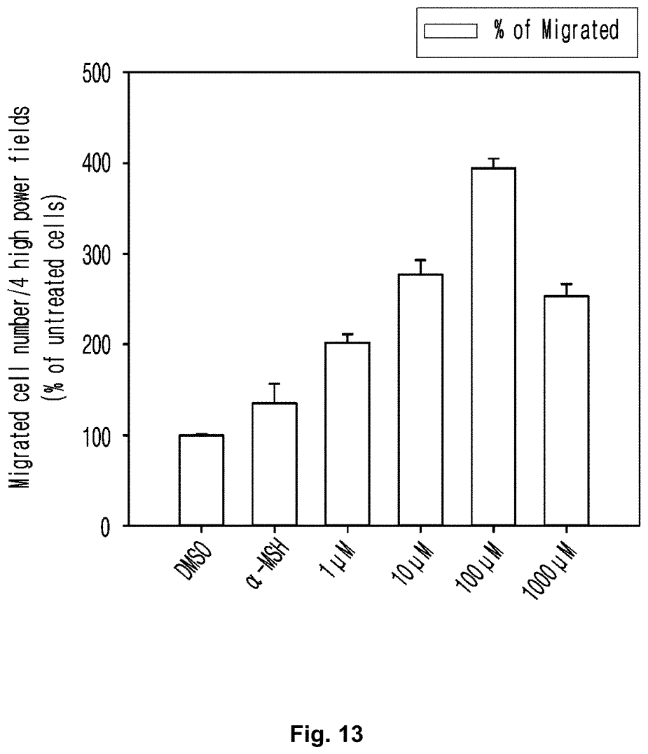

As shown in FIGS. 13 and 14 and Table 11, when the compound of Example 1 according to the present invention was treated at the concentrations of 1-100 μM, the cell migration rate was gradually increased compared to when the negative control was treated. Particularly, when the compound was treated at the concentration of 1 μM, the cell migration rate was increased by 2 times, when the compound was treated at the concentration of 10 μM, the cell migration was increased by 2.7 times, and when the compound was treated at the concentration of 100 μM, the cell migration rate was increased by about 4 times. On the other hand, when the compound was treated at the concentration of 1000 μM, the cell migration rate was decreased rather than when the compound was treated at the concentration of 100 μM.

<3-2> Confirmation of Cell Migration Effect According to Treatment Concentration of Compound of Example 1

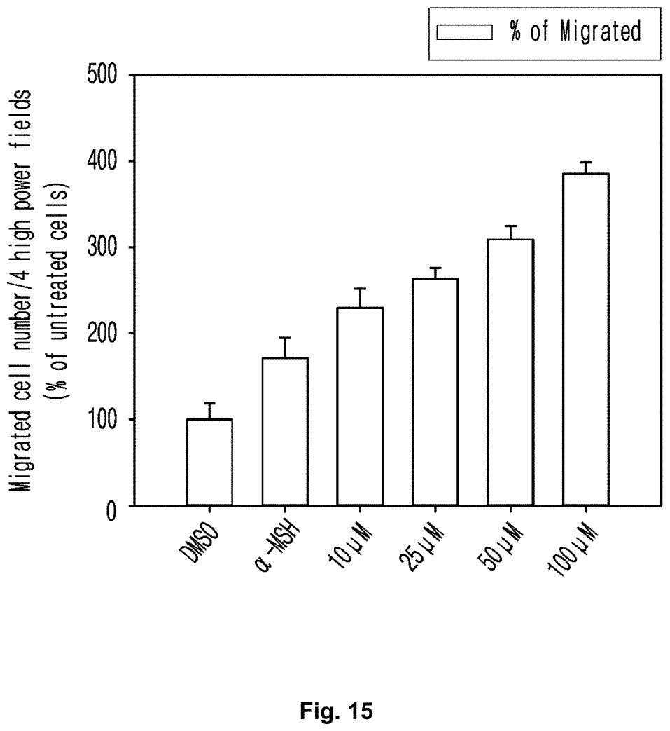

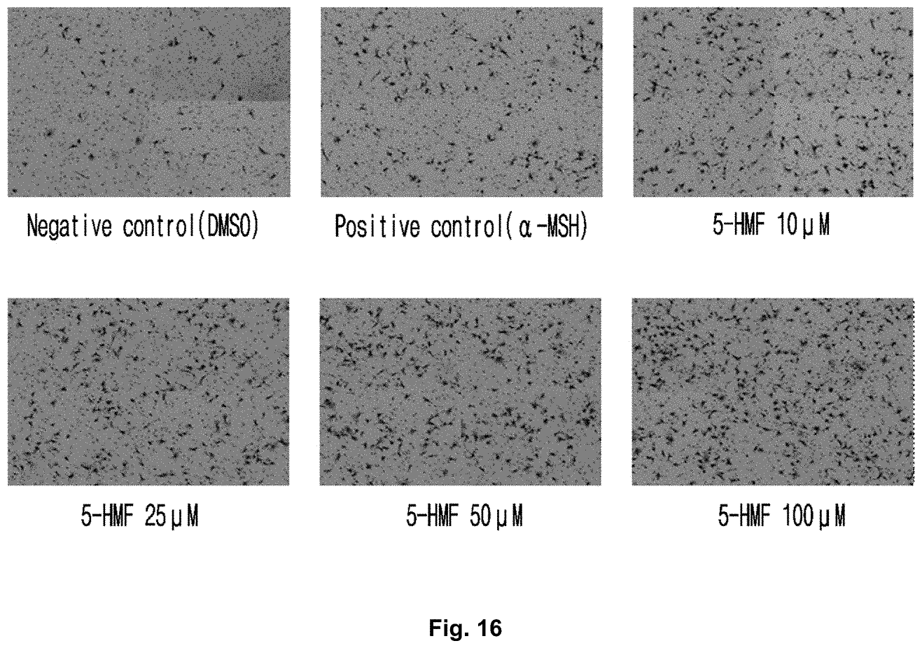

The treatment concentration of the compound of Example 1 was further subdivided, and the melanoblast migration effect according to the treatment of the compound was confirmed by performing the same method as described in Experimental Example <3-1>, and the results are shown in FIGS. 15 and 16 and Table 12.

TABLE 12

| | Cell migration rate (%) |
|---|---|
| Negative control (DMSO) | 100.00 ± 18.79 |
| Positive control (α-MSH) | 171.15 ± 23.70 |
| 10 μM | 229.49 ± 22.69 |
| 25 μM | 263.46 ± 12.65 |
| 50 μM | 308.97 ± 15.46 |
| 100 μM | 385.26 ± 13.13 |

As shown in FIGS. 15 and 16 and Table 12, when the compound of Example 1 according to the present invention was treated at the concentrations of 10-100 μM, the cell migration rate was gradually increased compared to when the negative control was treated.

<3-3> Confirmation of Cell Migration Effect According to Treatment of Compounds of Examples 2-10 at 10 μM

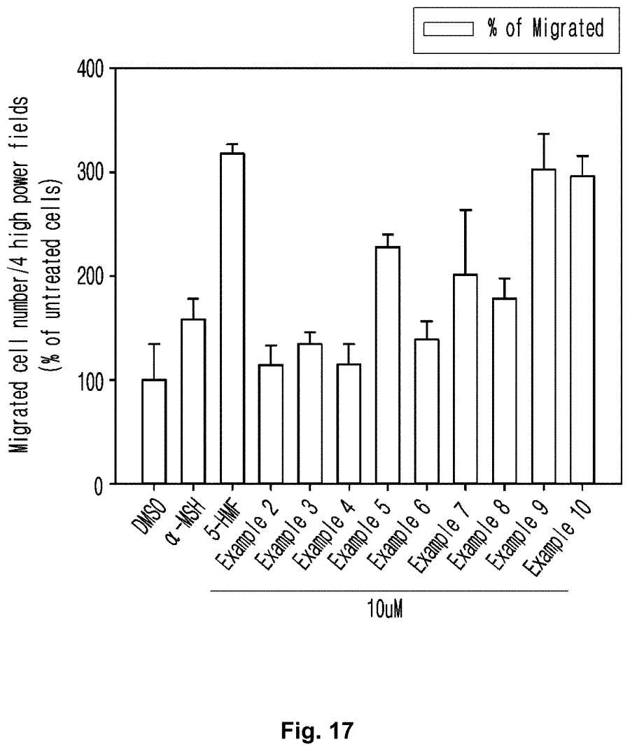

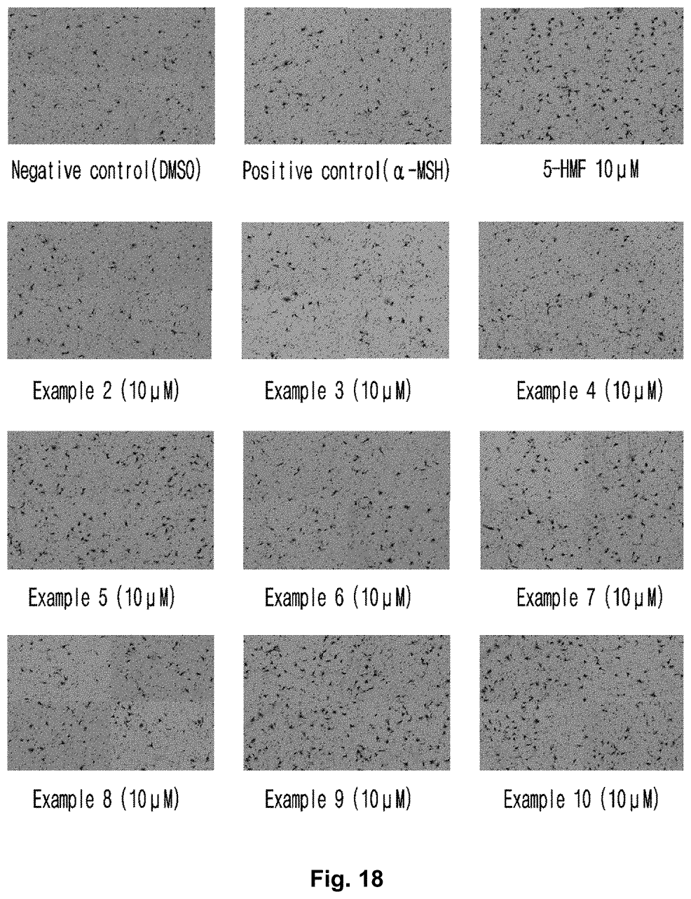

The melanoblast migration effect according to the treatment of the compounds of Examples 2-10 at the concentration of 10 μM was confirmed by performing the same method as described in Experimental Example <3-1>, and the results are shown in FIGS. 17 and 18 and Table 13.

TABLE 13

| | Cell migration rate (%) |
|---|---|
| Negative control (DMSO) | 100.00 ± 34.66 |
| Positive control (α-MSH) | 158.10 ± 20.08 |
| Example 1 10 μM | 318.10 ± 8.66 |
| Example 2 | 114.29 ± 18.86 |
| Example 3 | 134.29 ± 11.72 |
| Example 4 | 115.24 ± 19.50 |
| Example 5 | 228.57 ± 11.55 |
| Example 6 | 139.05 ± 17.51 |
| Example 7 | 201.90 ± 61.87 |
| Example 8 | 178.10 ± 19.80 |
| Example 9 | 302.95 ± 33.65 |
| Example 10 | 296.19 ± 19.42 |

As shown in FIGS. 17 and 18 and Table 13, when the compounds of Examples 2-10 according to the present invention were treated at the concentration of 10 μM, the cell migration rate was increased compared to when the negative control was treated. Particularly, when the compounds of Examples 5, 7, 9 and 10 were treated, the cell migration rate was increased more than 2 times. In particular, when the compound of Example 9 was treated, the cell migration rate was increased more than 3 times.

<3-4> Confirmation of Cell Migration Effect According to Treatment Concentration of Compounds of Examples 9 and 10

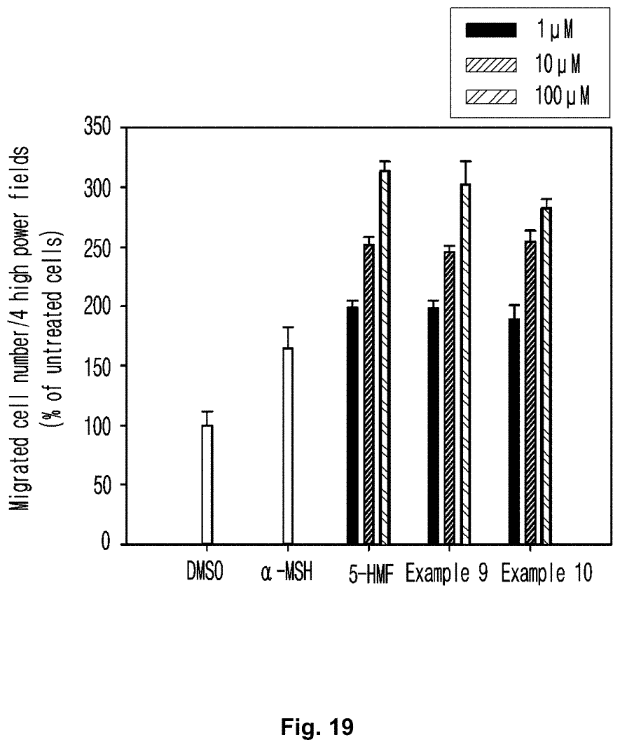

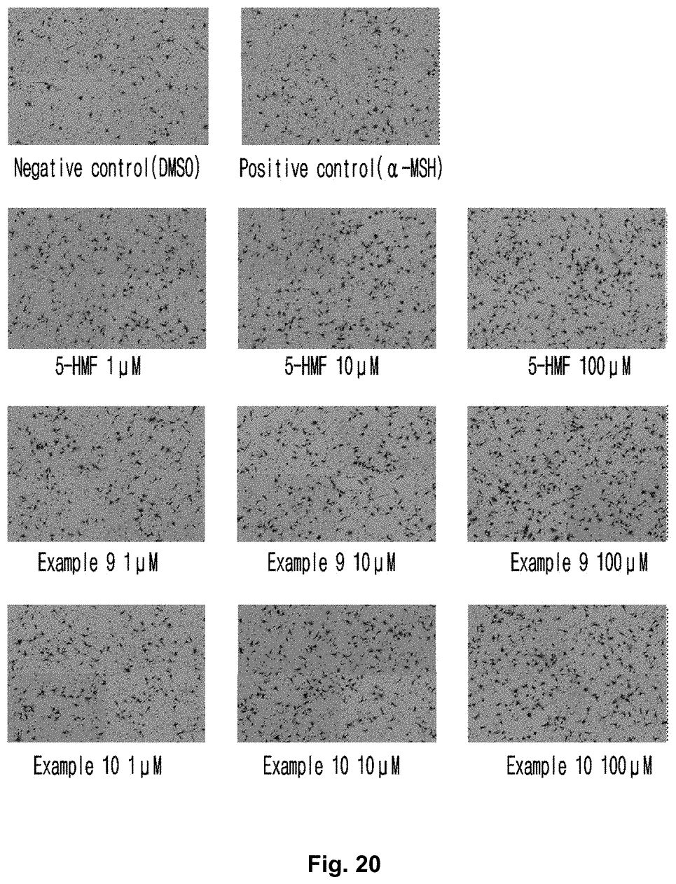

The melanoblast migration effect according to the treatment concentration of the compounds of Examples 9 and 10 was confirmed by performing the same method as described in Experimental Example <3-1>, and the results are shown in FIGS. 19 and 20 and Table 14.

TABLE 14

| | Cell migration rate (%) |
|---|---|
| Negative control (DMSO) | 100.00 ± 11.72 |
| Positive control (α-MSH) | 164.50 ± 18.12 |
| Example 1 1 μM | 198.82 ± 5.97 |
| Example 1 10 μM | 251.48 ± 6.86 |
| Example 1 100 μM | 313.61 ± 7.89 |
| Example 9 1 μM | 198.22 ± 6.81 |
| Example 9 10 μM | 246.15 ± 4.71 |
| Example 9 100 μM | 302.96 ± 18.68 |
| Example 10 1 μM | 189.35 ± 11.42 |
| Example 10 10 μM | 254.44 ± 8.99 |
| Example 10 100 μM | 282.84 ± 7.03 |

As shown in FIGS. 19 and 20 and Table 14, when the compounds of Examples 9 and 10 according to the present invention were treated at the concentrations of 1-100 μM, the cell migration rate was gradually increased compared to when the negative control was treated. Particularly, when the compounds were treated at the concentration of 1 μM, the cell migration rate was increased by about 1.9 times, and when the compounds were treated at the concentration of 10 μM, the cell migration was increased by about 2.5 times. In particularly, when the compound of Example 9 was treated at the concentration of 100 μM, the cell migration was increased by about 3 times, and when the compound of Example 10 was treated at the concentration of 100 μM, the cell migration was increased by about 2.7 times.

<3-5> Confirmation of Cell Migration Effect According to Treatment of Compounds of Examples 11-18 at 10 μM

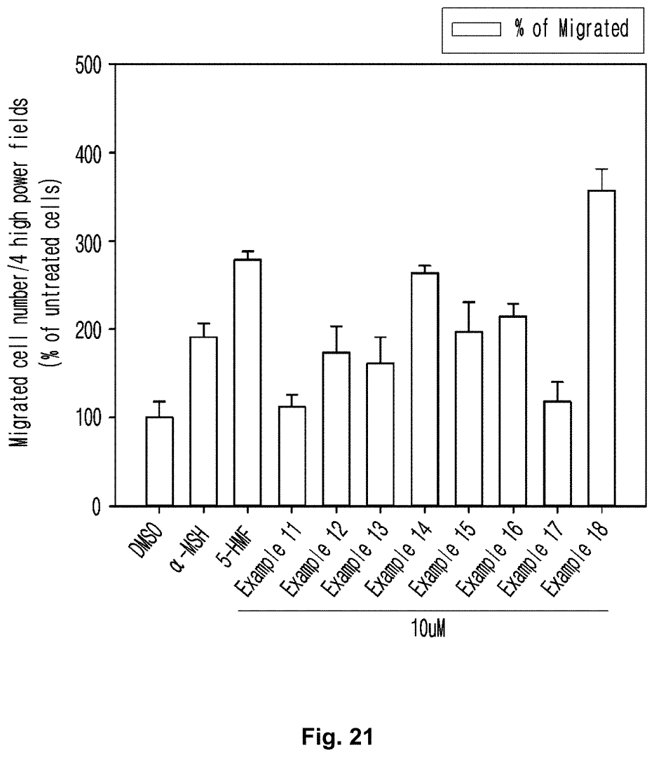

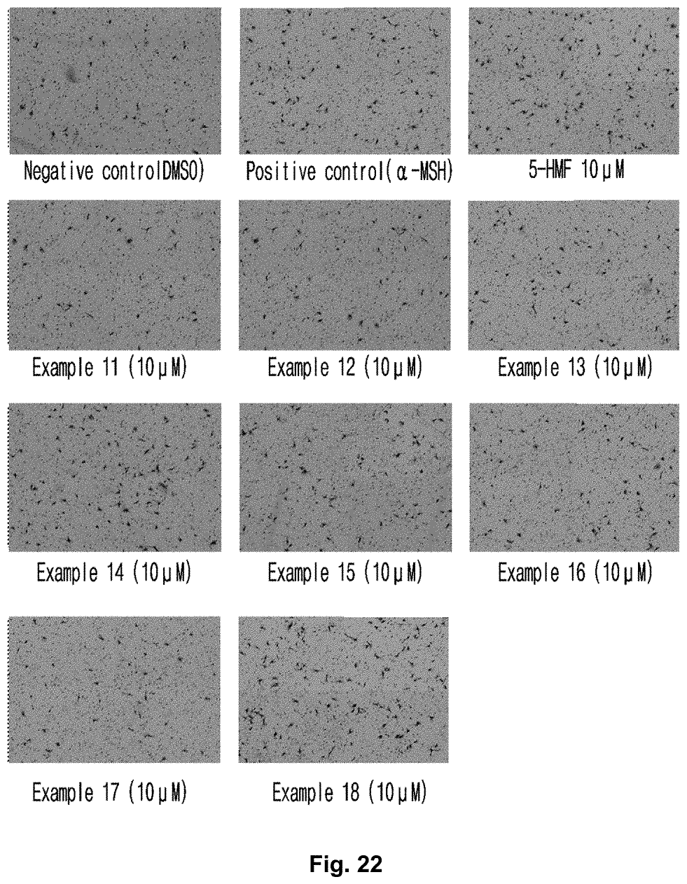

The melanoblast migration effect according to the treatment of the compounds of Examples 11-18 at the concentration of 10 μM was confirmed by performing the same method as described in Experimental Example <3-1>, and the results are shown in FIGS. 21 and 22 and Table 15.

TABLE 15

| | Cell migration rate (%) |
|---|---|
| Negative control (DMSO) | 100.00 ± 17.98 |
| Positive control (α-MSH) | 191.04 ± 15.31 |
| Example 1 10 μM | 279.10 ± 8.84 |
| Example 11 | 111.94 ± 13.89 |
| Example 12 | 173.13 ± 29.95 |
| Example 13 | 161.19 ± 29.36 |
| Example 14 | 264.18 ± 7.81 |
| Example 15 | 197.01 ± 33.82 |
| Example 16 | 214.93 ± 13.93 |
| Example 17 | 117.91 ± 21.95 |
| Example 18 | 356.72 ± 24.88 |

As shown in FIGS. 21 and 22 and Table 15, when the compounds of Examples 11-18 according to the present invention were treated at the concentration of 10 μM, the cell migration rate was increased compared to when the negative control was treated. Particularly, when the compounds of Examples 14, 16 and 18 were treated, the cell migration rate was increased more than 2 times. In particular, when the compound of Example 18 was treated, the cell migration rate was increased more than 3.5 times.

<3-6> Confirmation of Cell Migration Effect According to Treatment Concentration of Compound of Example 18

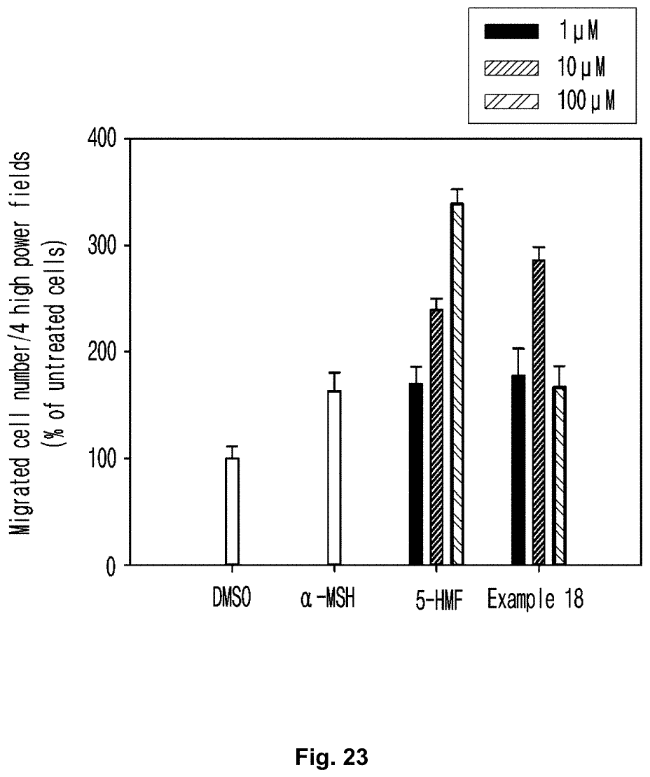

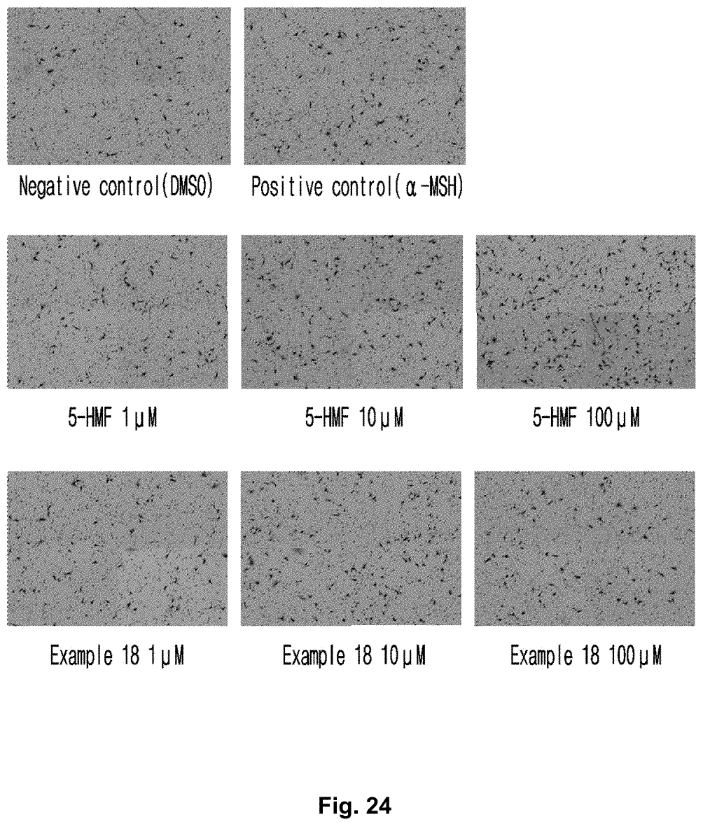

The melanoblast migration effect according to the treatment concentration of the compound of Example 18 was confirmed by performing the same method as described in Experimental Example <3-1>, and the results are shown in FIGS. 23 and 24 and Table 16.

TABLE 16

| | Cell migration rate (%) |
|---|---|
| Negative control (DMSO) | 100.00 ± 11.20 |
| Positive control (α-MSH) | 163.44 ± 17.34 |
| Example 1 1 μM | 169.89 ± 15.72 |
| Example 1 10 μM | 239.78 ± 10.23 |
| Example 1 100 μM | 338.71 ± 13.64 |
| Example 18 1 μM | 177.42 ± 25.52 |
| Example 18 10 μM | 286.02 ± 12.63 |
| Example 18 100 μM | 166.67 ± 20.26 |

As shown in FIGS. 23 and 24 and Table 16, when the compound of Example 18 according to the present invention was treated at the concentrations of 1-10 μM, the cell migration rate was gradually increased compared to when the negative control was treated. Particularly, when the compound was treated at the concentration of 1 μM, the cell migration rate was increased by about 1.7 times, and when the compound was treated at the concentration of 10 μM, the cell migration was increased by about 2.8 times. On the other hand, when the compound was treated at the concentration of 100 μM, the cell migration rate was rather decreased.

<3-7> Confirmation of Cell Migration Effect According to Treatment of Compounds of Examples 19-21 at 10 μM

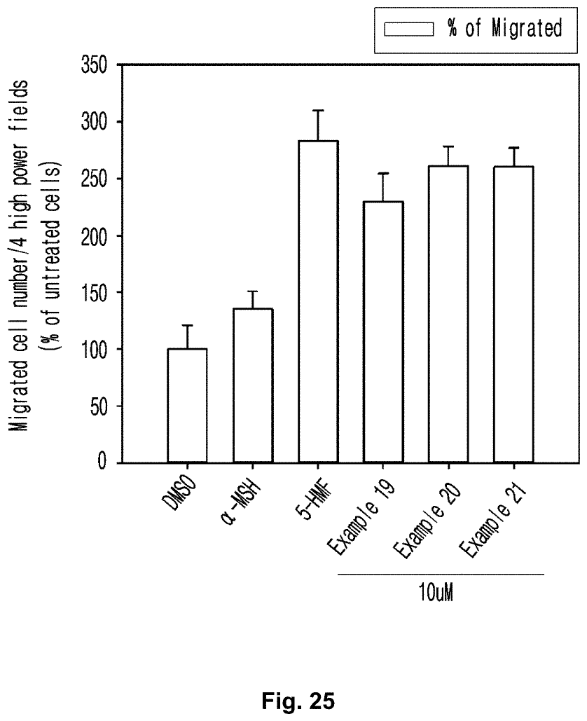

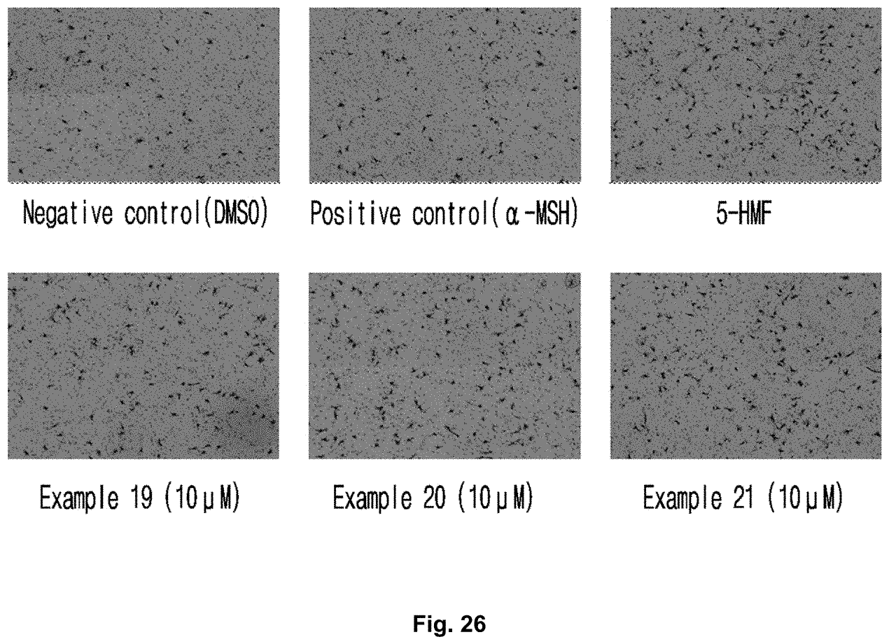

The melanoblast migration effect according to the treatment of the compounds of Examples 19-21 at the concentration of 10 μM was confirmed by performing the same method as described in Experimental Example <3-1>, and the results are shown in FIGS. 25 and 26 and Table 17.

TABLE 17

| | Cell migration rate (%) |
|---|---|
| Negative control (DMSO) | 100.00 ± 20.71 |
| Positive control (α-MSH) | 134.65 ± 16.34 |
| Example 1 | 283.17 ± 26.86 |
| Example 19 | 229.70 ± 25.07 |
| Example 20 | 261.39 ± 16.83 |
| Example 21 | 260.40 ± 16.54 |

As shown in FIGS. 25 and 26 and Table 17, when the compounds of Examples 19-21 according to the present invention were treated at the concentration of 10 μM, the cell migration rate was increased more than 2 times compared to when the negative control was treated.

From the above results, it was confirmed that the compound represented by formula 1 according to the present invention promoted and increased the migration of melanoblasts, and ultimately, increased the intracellular cell migration effect. Therefore, the compound represented by formula 1 according to the present invention can be effectively used for preventing, alleviating or treating poliosis or vitiligo.

The compound represented by formula 1 according to the present invention increases intracellular melanin content, accelerates or increases melanoblast migration, pre-emptively prevents gray hair formation, and accelerates dark hair formation, and thus may be beneficially used in the prevention or alleviation of gray hair, and in the prevention, alleviation or treatment of poliosis or vitiligo.

### INDUSTRIAL APPLICABILITY

The pharmaceutical composition of the present invention increases intracellular melanin content, accelerates or increases melanoblast migration, pre-emptively prevents gray hair formation, and accelerates dark hair formation, and thus may be beneficially used in the prevention or alleviation of greying of hair, and in the prevention, alleviation or treatment of poliosis or vitiligo.

1-12. (canceled)

13. A pharmaceutical, cosmetic or health food composition for preventing or alleviating poliosis, vitiligo or gray hair comprising a compound represented by formula 1 below, an isomer thereof, a solvate thereof, a hydrate thereof or a pharmaceutically acceptable salt thereof as an active ingredient: 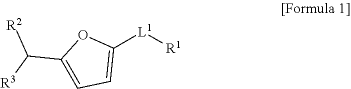
In formula 1,
L¹ is —C(═O)—, straight or branched Cis alkylene, —C(═O)O— or —C(═O)NH—, R¹ is hydrogen, OH, straight or branched C₁₋₅ alkyl, straight or branched C₁₋₅ alkylcarbonyloxy, C₆₋₁₀ aryl-C₁₋₂ alkyl, allyloxy or C₆₋₁₀ aryl-C₁₋₂ alkyloxy; and
R² and R³ are independently hydrogen, OH, straight or branched C₁₋₅ alkyl or straight or branched C₁₋₅ alkoxy, or, R² and R³ can form carbonyl (C═O) along with the carbon atom to which they are attached, except when R² and R³ are hydrogen at the same time.

14. The composition according to claim 1, wherein the L¹ in formula 1 is —C(═O)—, straight or branched C₁₋₃ alkylene, —C(═O)O— or —C(═O)NH—, R¹ is hydrogen, OH, straight or branched C₁₋₃ alkyl, straight or branched C₁₋₃ alkylcarbonyloxy, phenyl-C₁₋₂ alkyl, allyloxy or phenyl-C₁₋₂ alkyloxy; and
R² and R³ are independently hydrogen, OH, straight or branched C₁₋₃ alkyl or straight or branched C₁₋₃ alkoxy, or, R² and R³ can form carbonyl (C═O) along with the carbon atom to which they are attached, except when R² and R³ are hydrogen at the same time.

15. The composition according to claim 1, wherein the L′ in formula 1 is —C(═O)—, —CH₂—, —C(═O)O— or —C(═O)NH—,
R¹ is hydrogen, OH, methyl, ethyl, methylcarbonyloxy, benzyl, allyloxy or benzyloxy; and
R² and R³ are independently hydrogen, OH, methyl or methoxy, or, R² and R³ can form carbonyl (C═O) along with the carbon atom to which they are attached, except when R² and R³ are hydrogen at the same time.

16. The composition according to claim 1, wherein the compound represented by formula 1 is selected from the group consisting of the following compounds:
<1> 5-(hydroxymethyl)furan-2-carbaldehyde;
<2> 5-((benzyloxy)methyl)furan-2-carbaldehyde;
<3> 5-((allyloxy)methyl)furan-2-carbaldehyde;
<4> N-benzyl-5-formylfuran-2-carboxamide;
<5> 5-formyl-N-propylfuran-2-carboxamide;
<6> 5-formyl-N-methylfuran-2-carboxamide;
<7> benzyl 5-formylfuran-2-carboxylate;
<8> allyl 5-formylfuran-2-carboxylate;
<9> methyl 5-(dimethoxymethyl)furan-2-carboxylate;
<10> methyl 5-formylfuran-2-carboxylate;
<11> ethyl 5-(hydroxymethyl)furan-2-carboxylate;
<12> furan-2,5-diyldimethanol;
<13> methyl 5-(hydroxymethyl)furan-2-carboxylate;
<14> 5-(hydroxymethyl)furan-2-carboxylic acid;
<15> 1-(5-(((tetrahydro-2H-pyran-2-yl)oxy)methyl)furan-2-yl)ethanol;
<16> 1-(5-(hydroxymethyl)furan-2-yl)ethanol;
<17> furan-2,5-dicarbaldehyde;
<18> 5-(1-hydroxyethyl)furan-2-carbaldehyde;
<19> 1-(5-formylfuran-2-yl)ethyl acetate;
<20> 5-(1-hydroxypropyl)furan-2-carbaldehyde; and
<21> 1-(5-formylfuran-2-yl)propyl acetate.

17. The composition according to claim 1, wherein the compound represented by formula 1 increases intracellular melanin content in a subject.

18. The composition according to claim 1, wherein the compound represented by formula 1 promotes migration or differentiation of melanoblasts in a subject.

19. The composition according to claim 1, wherein the composition is administered orally to the subject in the form of a tablet, pill, capsule, solution, syrup, granule, elixir, or troche.

20. The composition according to claim 1, wherein the composition is externally applied to the skin of the subject as an ointment, paste, cream, gel, liquid, suspension, or emulsion.

21. The composition according to claim 1, wherein the composition is administered parenterally as an aqueous solution, water-insoluble excipient, suspension, or emulsions to the subject.
